(19) United States
(12) Patent Application Publication
(10) Pub. No.: US 2022/0178896 A1
(43) Pub. Date: Jun. 9, 2022

(54) CONSUMER PRODUCTS COMPRISING CROSS-LINKED CARBON NANOTUBE SENSORS AND SYSTEMS AND METHODS COMPRISING THE SAME

(71) Applicant: The Procter & Gamble Company, Cincinnati, OH (US)

(72) Inventors: Martin James PEACOCK, Newcastle upon Tyne (GB); Dana Paul GRUENBACHER, Fairfield, OH (US); Eric San Jose ROBLES, Newcastle upon Tyne (GB); Rebecca Louise WOOD, North Shields (GB); Milo Sebastian Peter SHAFFER, Reading (GB); David Mark STRINGER, London (GB)

(21) Appl. No.: 17/543,765

(22) Filed: Dec. 7, 2021

Related U.S. Application Data
(60) Provisional application No. 63/122,499, filed on Dec. 8, 2020.

(51) Int. Cl. G01N 33/00; C01B 32/174

(52) U.S. Cl. CPC G01N 33/0062; C01B 32/174; G01N 2033/0068; G01N 33/0031; C01B 2202/22; C01P 2006/40; C01B 2202/08; C01B 2202/02

(57) ABSTRACT

A consumer product having a sensor for controlling the operation of the consumer product, a system and method including the consumer product and a sensor are provided. The system and method including a central communication unit capable of receiving incoming signals and sending outgoing instructions from the consumer product and sensor. The central communication unit communicably connected with a memory configured to store an algorithm. The sensor has a cross-linked carbon nanotube network comprising: a plurality of carbon nanotubes; and at least one linker that covalently links adjacent carbon nanotubes. The algorithm controls the consumer product based on incoming signals sent from the sensor to the central communication unit.

### FIELD

The present disclosure relates to consumer products comprising cross-linked carbon nanotube sensors and systems and methods comprising the same.

### BACKGROUND

The mass availability of data is changing the way people live their lives, with huge advantages arising from the capability to monitor the gases present in surrounding environments, both industrially and at home. There is, therefore, now a well-demonstrated need for gas sensor devices that achieve high sensitivity and selectivity, yet have small dimensions, low-power requirements and an affordable manufacturing route.

Further, people are using data to automate the operation of various consumer products and appliances in their homes and businesses. Thus, there is a need to couple gas sensor devices that achieve high sensitivity and selectivity with consumer products that are able to mitigate or boost the level of a target gas in a space, including systems and methods comprising the same.

### SUMMARY

### Combinations:

A. A system for utilizing a consumer product, the system comprising:
a central communication unit capable of receiving incoming signals and sending outgoing instructions, the central communication unit communicably connected with a memory configured to store an algorithm;
a sensor communicably connectable with the central communication unit and configured to send incoming signals to the central communication unit alerting the central communication unit of an identification and concentration of a target gas of interest, the sensor comprising a cross-linked carbon nanotube network comprising: a plurality of carbon nanotubes; and at least one linker that covalently links adjacent carbon nanotubes; and
a consumer product capable of boosting or mitigating the target gas of interest.
   B. The system of Paragraph A, wherein the consumer product is communicably connectable with the central communication through a wireless communication link, wherein the algorithm controls the consumer product using incoming signals sent from the sensor to the central communication unit.
   C. The system of Paragraph A or Paragraph B, wherein the consumer product is selected from the group consisting of: an air freshener device, an air cleaning device, a tooth brush, a razor, a diaper, a feminine care product, a cleaning implement, and combinations thereof.
   D. The system of any of Paragraphs A through C, wherein the plurality of carbon nanotubes are single-walled carbon nanotubes.
   E. The system of any of Paragraphs A through D, wherein the plurality of carbon nanotubes are linked in a series of parallel layers, optionally wherein the cross-linked carbon nanotube has an in-plane conductivity that is greater than the through-thickness conductivity.
   F. The system of any of Paragraphs A through E, wherein the linker is a conjugated linker having a moiety of structure *-A-*, wherein A is a divalent conjugated system comprising one or more aryl or heteroaryl rings and * is the point of attachment to the carbon nanotubes.
   G. The system of Paragraph F, wherein A is:

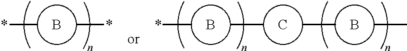

wherein
each Ring B is independently an optionally substituted aryl or heteroaryl;
Ring C is an optionally substituted porphyrin ring;
n is an integer from 1 to 5; and
* indicates the point of attachment to each X;
   optionally wherein each Ring B may independently be

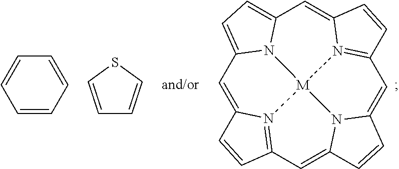

each of which may be optionally substituted, and wherein M is Zn, Cu, Ni or Co.
H. The system of Paragraph G, wherein the one or more aryl or heteroaryl rings are substituted with one or more of C₁-C₂₀alkyl, amino (including alkylamino and dialkylamino), carboxylic acid, amino-C₁-C₆alkyl, C₁-C₆alkyl-COOH, and —NHC(S)(NH₂), or a substituent comprising a Noble metal nanoparticle, a porphyrin, a calix[4]arenes or a crown ether.
I. The system of any of Paragraphs A through H, wherein the linker is a conjugated linker having a moiety of structure:

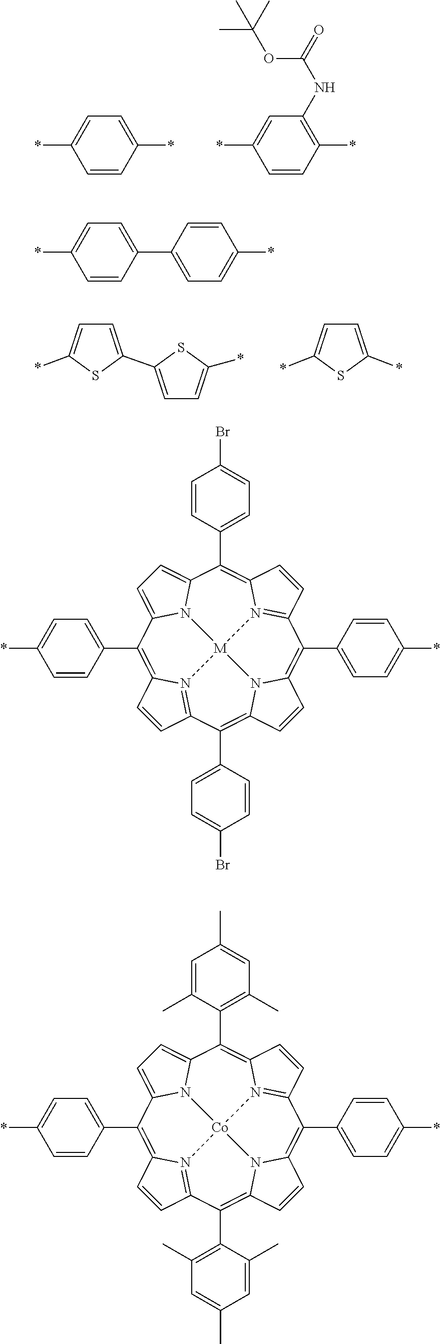

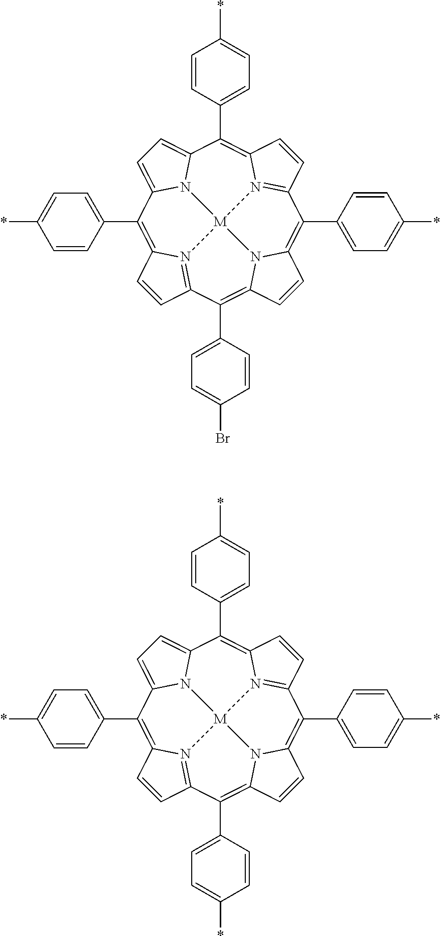

wherein M is Zn, Cu, Ni or Co.
J. The system of any of Paragraphs A through I, wherein the linker is a rigid linker having a moiety of structure *—Y—*, wherein Y is a multivalent rigid system and * is the point of attachment to the carbon nanotubes.
K. The system of Paragraph J, wherein Y comprises a cycloalkyl group or a polyoctahedral silsequioxane.
L. The system of any of Paragraphs A through K, wherein the plurality of carbon nanotubes form a film, wherein the film is provided on a substrate.
M. The system of Paragraph L, wherein the film has a thickness of about 1 to about 500 nm.
N. The system of any of Paragraphs A through M, wherein the wireless communication link is selected from the group consisting of: Wi-Fi; Bluetooth; ZigBee, 6LoWPAN, Thread, Mesh Network, or combinations thereof.
O. A consumer product comprising a sensor, the sensor comprising a cross-linked carbon nanotube network comprising: a plurality of carbon nanotubes; and at least one linker that covalently links adjacent carbon nanotubes.
P. The consumer product of Paragraph O, wherein the consumer product is selected from the group consisting of: an air freshener device, an air cleaning device, a tooth brush, a razor, a diaper, a feminine care product, a cleaning implement, and combinations thereof.
Q. The consumer product of Paragraph O or Paragraph P, wherein the plurality of carbon nanotubes are single-walled carbon nanotubes.
R. The consumer product of any of Paragraphs O through Q, wherein the plurality of carbon nanotubes are linked in a series of parallel layers, optionally wherein the cross-linked carbon nanotube has an in-plane conductivity that is greater than the through-thickness conductivity.
S. The consumer product of any of Paragraphs O through R, wherein the linker is a conjugated linker having a moiety of structure *-A-*, wherein A is a divalent conjugated system comprising one or more aryl or heteroaryl rings and * is the point of attachment to the carbon nanotubes.
T. The consumer product of Paragraph S, wherein A is:

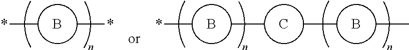

wherein
each Ring B is independently an optionally substituted aryl or heteroaryl;
Ring C is an optionally substituted porphyrin ring;
n is an integer from 1 to 5; and
* indicates the point of attachment to each X;
   optionally wherein each Ring B may independently be

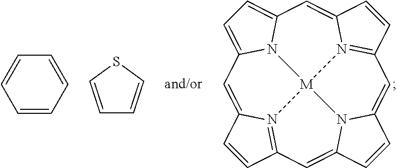

each of which may be optionally substituted, and wherein M is Zn, Cu, Ni or Co.
U. The consumer product of Paragraph T, wherein the one or more aryl or heteroaryl rings are substituted with one or more of C₁-C₂₀alkyl, amino (including alkylamino and dialkylamino), carboxylic acid, amino-C₁-C₆alkyl, C₁-C₆alkyl-COOH, and —NHC(S)(NH₂), or a substituent comprising a Noble metal nanoparticle, a porphyrin, a calix[4]arenes or a crown ether.
V. The consumer product of any of Paragraphs O through U, wherein the linker is a conjugated linker having a moiety of structure:

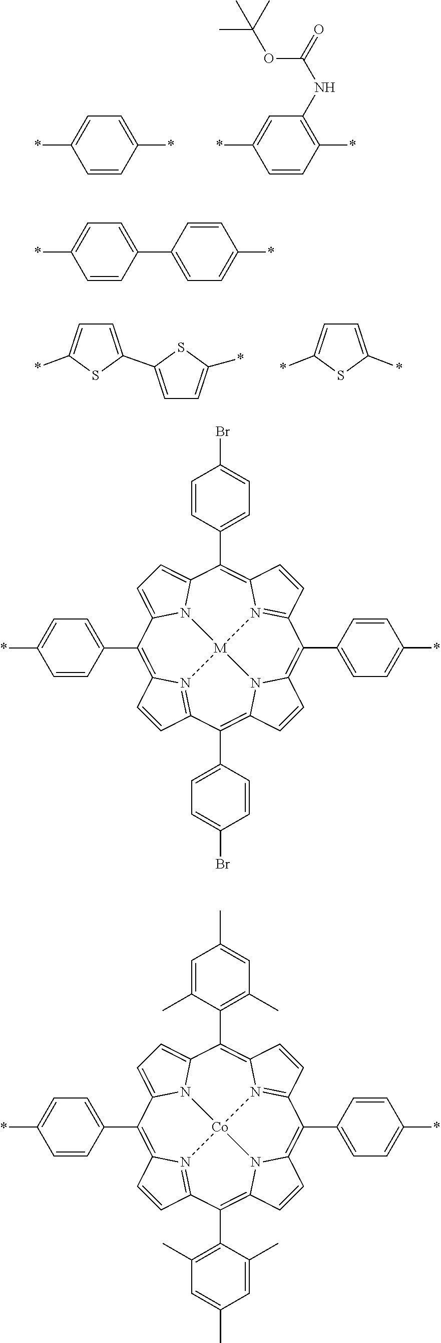

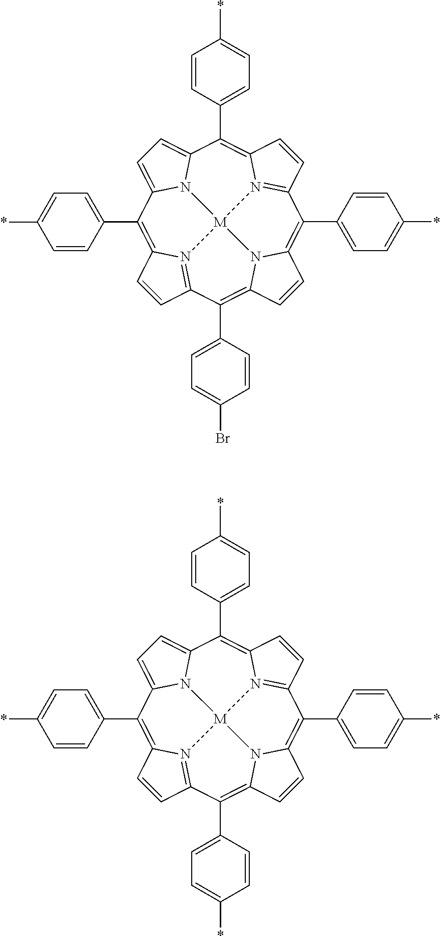

wherein M is Zn, Cu, Ni or Co.
W. The consumer product of any of Paragraphs O through V, wherein the linker is a rigid linker having a moiety of structure *—Y—*, wherein Y is a multivalent rigid system and * is the point of attachment to the carbon nanotubes.
X. The consumer product of Paragraph W, wherein Y comprises a cycloalkyl group or a polyoctahedral silsequioxane.
Y. The consumer product of any of Paragraphs O through X, wherein the plurality of carbon nanotubes form a film, wherein the film is provided on a substrate.
Z. The consumer product of Paragraph Y, wherein the film has a thickness of about 1 to about 500 nm.
AA. A method of controlling the operation of a consumer product based on incoming signals from a sensor, wherein the incoming signals identify a target gas the concentration of the target gas, wherein the sensor and the consumer product are each communicably connectable with a central communication unit, the central communication unit communicably connected with a memory capable of storing set points, wherein the sensor comprising a cross-linked carbon nanotube network comprising: a plurality of carbon nanotubes; and at least one linker that covalently links adjacent carbon nanotubes, wherein the linker is comprises a conjugated system directly conjugated to the carbon nanotubes, the method comprising the steps of:
setting a concentration set point in the memory for a target gas;
identifying and measuring the concentration of the target gas with the sensor;
sending an incoming signal from the sensor to the central communication unit, the incoming signal comprising a concentration measurement of the target gas;
comparing the concentration measurement with the concentration set point for the target gas;
identifying one or more consumer products to mitigate or boost the target gas.
   BB. The method of Paragraph AA further comprising the step of adjusting the operation of the consumer product if the concentration measurement is different than the concentration set point for the target gas.
   CC. The method of Paragraph AA or Paragraph BB, wherein the sensor is configured as an array capable of identifying and measuring a plurality of target gases.
   DD. The method of any of Paragraphs AA through CC further comprising the step of notifying a user on a consumer product usage capable of boosting or mitigating the target gas.
   EE. The method of any of Paragraphs AA through DD, wherein the central communication unit communicates with two or more consumer products, the method further comprising the steps of:
setting a second concentration set point in the memory for a second target gas that is different from the first target gas;
identifying and measuring the concentration of the second target gas with the sensor;
sending a second incoming signal from the sensor to the central communication unit, the incoming signal comprising a second concentration measurement of the second target gas;
comparing the second concentration measurement with the second concentration set point for the target gas;
sending a second outgoing instruction to the consumer product to turn ON, OFF, or adjust the operation of a second consumer product if the second concentration measurement is different than the second concentration set point for the second target gas.

### BRIEF DESCRIPTION OF THE DRAWINGS

FIG. 1 illustrates a system having a central communication unit, a sensor, and a consumer product.

FIG. 2 illustrates the placement of a consumer product and sensor in a different room from the placement of a CCU.

FIG. 3 illustrates multiple possible flows of sensor measurements.

FIG. 4 shows a schematic of single walled carbon nanotube (a) reductive dissolution and crosslinking with a conjugated linker precursor and (b) molecular layer deposition with alternate layers of carbon nanotube and linker.

FIG. 5 illustrates an exemplary CCU having the processor and the memory disposed within a housing.

FIG. 6 illustrates multiple exemplary flows of incoming signals to a remote memory.

FIG. 7 illustrates an exemplary algorithm that couples the operation of a consumer product and/or smart device with measurements from a sensor(s).

FIG. 8 illustrates an exemplary system having more than one user interface.

FIG. 9 shows (a) a schematic representation of the cross-linking and deposition process and (b) a digital photograph of aerogel film deposited on glass substrate. (c) UV-Vis spectra of aerogel thin film, (d,e) atomic force microscopy height image of aerogel film scar edge and corresponding height profile.

FIG. 10 shows (a) statistical Raman spectroscopy, (b) thermogravimetric analysis of monolithic aerogel.

FIG. 11 shows (a) full XPS survey of as-received HiPco SWCNTs and aerogel thin films, (b) high resolution scan of SWCNT-Aniline and (c) SWCNT-Phenyl C1s peaks and corresponding peak fittings. (d) high-resolution scan of N1s peak of SWCNT-Aniline aerogel film and (e) I3d peak of SWCNT-Phenyl.

FIG. 12 shows N2 adsorption/desorption isotherms. (a) raw SWCNT powder and (b-d) crosslinked aerogel monoliths (Pore size distributions inset).

FIG. 13 shows (c) scanning electron micrographs of aerogel films without and with (d,e) crosslinking.

FIG. 14 shows Helium-ion microscopy of SWCNT thin films consisting of a) 1 layer, b) 2 layers without crosslinking and (c) 2 layers with a reductive crosslinking step.

FIG. 15 shows pixel thresholding of Helium-ion microscopy images.

FIG. 16 shows AFM images and corresponding height profiles of a) single-layer, b) double layer (non-crosslinked) and c) crosslinked double layer films.

FIG. 17 shows UV-Vis absorption of single and double layer films, b) multilayer films from 1-10 layers and c) the corresponding plot of transmittance vs layer number.

FIG. 18 shows single point Raman spectra of SWCNT films and b) statistical analysis of 25-point Raman map scans.

FIG. 19 shows the response of a cross-linked carbon nanotube network to different volatile amines: (a) ammonia, (b) DMEA, (c) TEA. Shaded regions indicate exposure to gas analyte.

FIG. 20 shows (a) sensor response to AcOH at high concentration, (b) response to AcOH at lower concentration and (c) IVA in ppb regime.

FIG. 21 shows dynamic range resistance change against concentration for each gas—dynamic range.

FIG. 22 shows (left) a schematic illustration of aniline-crosslinked MND film and (right) linear range and sensing response (inset) of a 6 layer film to increasing concentration of AcOH vapour, with the theoretical limit of detection (LoD) indicated.

### DETAILED DESCRIPTION

While the consumer products, systems, and methods of the present disclosure will be described more fully it is to be understood at the outset of the description which follows that persons of skill in the appropriate arts may modify the methods and systems herein described while still achieving the favorable results of described in the present disclosure. Accordingly, the description which follows is to be understood as being a broad, teaching disclosure directed to persons of skill in the appropriate arts, and not as limiting upon the present disclosure.

The system, methods, and devices of the present invention include a highly selective and sensitive sensor. The sensor may be used to detect the presence of a target gas. The target gas may be an undesirable gas that a user may wish to mitigate by either masking, removing, or reducing the presence of with one or more consumer products, such as an air freshener device or an air cleaning device. The target gas may be a desirable gas, such as a volatile perfume raw material, that a user may wish to boost or increase the presence of with a consumer product, such as an air freshener device. The sensor may be integral with the consumer product or may exist as a separate component that wirelessly communicates with a controller, a user or user interface, and/or a consumer product.

The consumer product may be a smart consumer product capable of communicating with a CCU or directly with the sensor. The consumer product may also require user intervention once the sensor informs the user that a condition has been met.

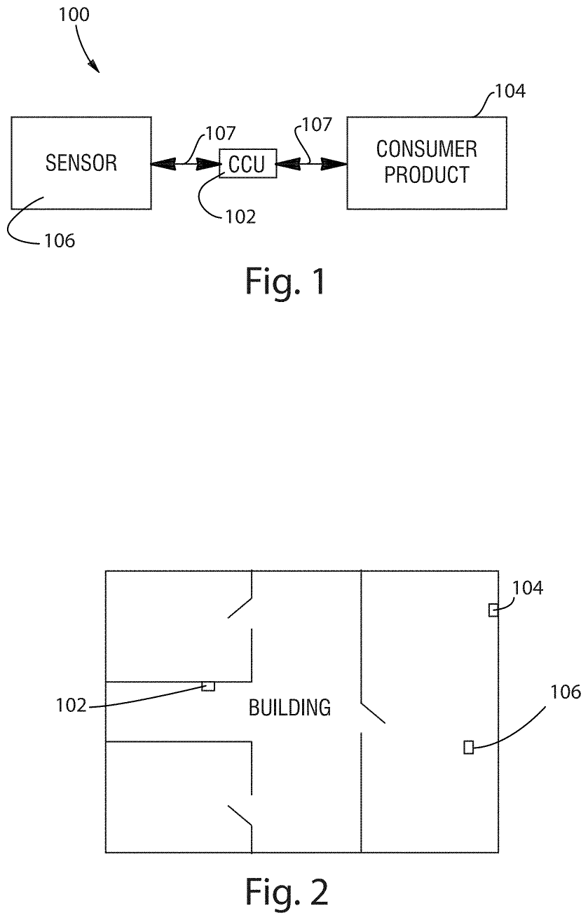

The systems and methods may include a central communication unit (CCU) that is communicably connectable with a consumer product(s) 104 and/or one or more sensors. The CCU may be in the form of a smart phone, computer, tablet, thermostat, and the like. FIG. 1 illustrates an exemplary, non-limiting, system 100, including the central communication unit 102, a consumer product(s) 104, and a sensor(s) 106 that each communicate with the CCU 102 through a wireless communication link 107.

For example, the sensor 106 may send incoming signals to the CCU 102. In response to the incoming signals from the sensor 106, the CCU may send outgoing instructions to the consumer product 104 to turn the consumer product ON or OFF or to adjust the operation of the consumer product. For example, if the sensor 106 sends an incoming signal on the presence and concentration of a particular target gas to the CCU, and the CCU compares the value of the incoming signal on the target gas to a setpoint for that particular target gas, the CCU may send an outgoing instruction to the consumer product 104 in order to mitigate or boost the level of that particular target gas, depending on whether the value of the incoming signal was higher or lower than the setpoint for the target gas.

As discussed in more detail below, the CCU may comprise a memory that is capable of storing set points and algorithms and a processor that is capable of running algorithms and accessing the stored set points from the memory. Various algorithms may be programmed depending upon the desired outcome.

The sensor(s) and consumer product(s) may be placed in any location within or outside of a building. The sensor(s) and consumer product(s) may be located in the same room as the other components of the system the components of the system may be disposed in one or more different rooms. With reference to FIG. 2, in a non-limiting illustrative example, the consumer product 104 may be placed in the same room as the sensor 106 and may be placed in a different location than the CCU 102. The consumer product 104 and/or the sensor 106 may be movable to different rooms at the user's convenience. The system may include a plurality of sensors that are positioned in various locations or rooms in a building. The system may also include a plurality of consumer products.

The system may also include additional smart devices 109 located in a building, such as appliances (refrigerator, washer, dryer, stove, microwave), fans, lights, electrical outlets, switches, televisions, HVAC systems, speakers, security systems, baby monitors, garage door openers, doorbells, cameras, wearables such as a smart watch or baby sleep monitor, and the like.

Sensors

The system 100 may include a sensor 106. The sensors 106 may be configured to measure the presence and concentration of target gases of interest. The target gases of interest may be undesirable target gases of interest that a user may wish to mitigate, or the target gases of interest may be desirable that a user may wish to boost or maintain at a desired concentration. It may be desirable to be able to detect and distinguish undesirable gases of interest from desirable gases of interest. Target gases of interest may include amines, including trimethyl amine (from, for example, urine, fish odor); aldehydes/ketones, such as 2-nonenal, nonanal, octanal, formaldehyde, 1-octen-3-one, and 3-octanone (from, for example, fragrance components); acids, such as acetic acid, isovaleric acid, and myristic acid (from, for example, body odors, cigarette/cigar smoke, bacon grease, and pet odors); alcohols, including 1-dodecanal, 1-octen-3-ol, 3-methyl-1-butanol, and 2-methylisoborneol (from, for example, mold/mildew); sulfur, including dimethyl disulfide (from, for example, trash, bathrooms, pets); aromatic hydrocarbons, including benzene, napthalene, and geosmin (from, for example, cigarette/cigar smoke and bacon grease); alkenes, including 1-pentadecene; lactones, including 5-H-furan-2-one, undecalactone; harmful air quality gases such as NO₂; and the like. Desirable target gases may include perfume raw materials such as aldehydes, ketones, esters, alcohols, and the like. Desirable target gases of interest may be used as a marker to measure the concentration of a perfume mixture in the air. That is, measuring a target gas of interest may be used to calculate a total concentration of a perfume mixture in the air.

The system may include sensors that also measure temperature, relative humidity, indoor air quality, outdoor air quality, noise, human presence, motion, air velocity in room, particle concentration in the air, allergens and/or other air borne entities that have effect on human health.

By combining the data from any and/or all combinations of these sensor sources as well as the target gas sensor or arrays of target gas sensors (multiple sensors each detecting unique target gases) it has been shown that a more accurate prediction may be made of the composition of the air. For example, by detecting people movement, hearing the sounds of cooking on stove along with rise in temp/humidity, along with unique target-gas sensing and particle sensing, we are more accurately able to predict when a person is cooking and exactly what smells or chemicals are in the air associated with the cooking based off the knowledge based algorithms stored. This sensor data can then give a user more accurate information or suggestions on how to manage a particular odor or air quality event. In another non-limiting example, detecting higher air flow in room as well as outdoor sounds combined with target gas and particle sensing can determine that a window is open and a particular event is happening (e.g. forest fire smoke) is coming through windows or a unique outdoor pollution(s) is entering the indoor space.

Specific sounds of interest that may be recognized with a sensor include, but are not limited to: cooking with stove, oven, or microwave, pets (e.g. barking), flushing a toilet, street noise (e.g. cars/traffic), people present and number of people, doors opening and closing, fan running, TV or radio, HVAC running. In particular, we have ability to recognize the presence or people or people talking but the algorithm can be designed to not actually use voice recognition or record what people are saying so as to protect privacy. This can be done by monitoring the types of sounds but not monitoring or deciphering what is said.

Another example, with the air flow sensing, we can determine if windows are open, doors are open, ceiling fans are running or when a HVAC unit or a room air circulating fan is operating. Further, outdoor air quality data for the home or location, such as data available from companies like Breezometer, can be leveraged to predict the outdoor air quality, pollen levels, temperature, humidity of the air outside any particular home or business.

When we combine these different sources of sensor data (e.g. noise, air flow, air quality) with the target gas sensor we can make a more informed recommendation to consumer or to another device on any corrective action needed. Non-limiting examples of corrective actions could be for consumer to turn on a kitchen vent, turn on an air cleaner, replace a filter of an air cleaner or HVAC, open windows, turn on a fan/HVAC, spray an air freshener, or any number of actions based on the various sensor data from all these sources, and combinations thereof. The sensor 106 may include a wireless communication module in order to be communicably connectable with the CCU and/or various components of the system through a wireless communication link.

The sensor 106 may be powered by a power source. The sensor 106 may be powered independently from the consumer product or through the same power source of the consumer product. The sensor 106 may be independently powered. The sensor may be battery powered or powered through an electrical outlet.

The sensor may be configured to send sensor measurements to the CCU in the form of incoming signals. The sensor measurements can be used in a variety of ways. For example, the sensor measurements may be viewed as live data; compared with set points, such as target gas concentration set points in order to control the air handling device; or stored in a database for further analysis to recommend optimum set points taking comfort and energy efficiency into consideration.

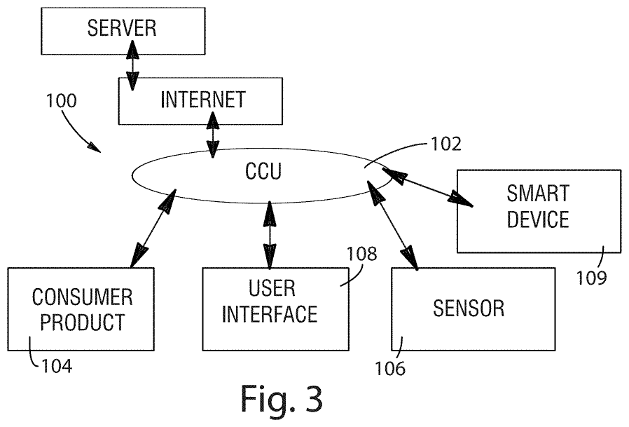

FIG. 3 depicts multiple possible flows of signals from the components to the CCU. Sensor measurements can flow from a component through the CCU to a user interface for live local sensor measurements. The sensor measurements may also pass from the sensor through the CCU to a destination server on the internet where it is stored in memory or analyzed by a processor in order to send instructions to the various components, including, but not limited to, the consumer product 104 and/or smart devices. The sensor measurements may also pass from the sensor through the CCU to the consumer product 104.

The sensor may be configured in various different ways. The sensor may be a stand-a-lone device or unit that is movable throughout a space. The sensor may be incorporated into another device, such as a consumer product or smart device.

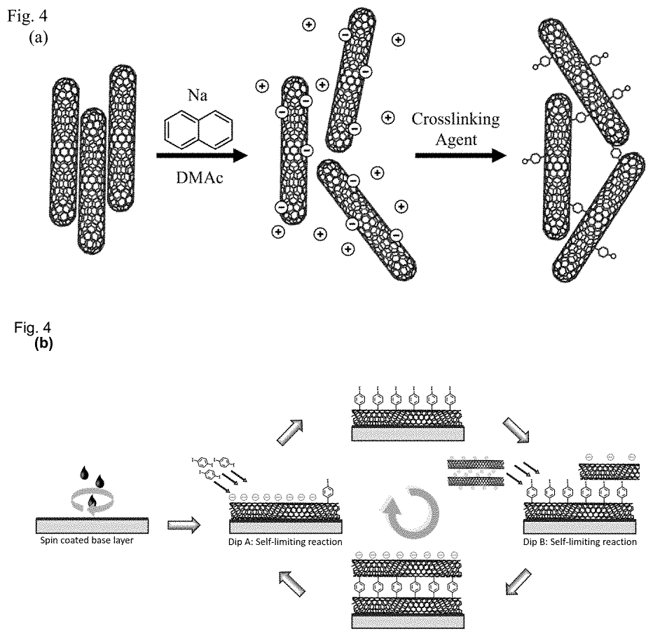

Reductive dissolution offers a route to highly individualized carbon nanotube solutions at high mass loading, without damaging the sp² π-system. (Clancy, A. J. et al. Charged Carbon Nanomaterials: Redox Chemistries of Fullerenes, Carbon Nanotubes, and Graphenes. Chem. Rev. 118, 7363-7408 (2018), the entire contents of which are herein incorporated by reference). Such solutions undergo grafting reactions with, for example, alky and aryl halides, allowing for the efficient attachment of binding sites. Biaryl halides, for example, may be utilised to crosslink adjacent carbon nanotubes, resulting in free-standing 3-dimensional networks that exhibit high-specific surface area with well-defined pore structures (FIG. 4).

As described herein, the cross-linking chemistry has been adapted to achieve cross-linked carbon nanotube aerogel thin films. The thin aerogel structure provides a sensor material that has a large active surface area with rapid access to the available porosity. Through this strategy, low limits of detection for both gases (such as volatile acids and amines) have been demonstrated.

It is hypothesised that the thin film aerogel architecture is ideally suited to gas sensing applications because of the high surface area and accessible porosity.

Additional enhancement may be imparted through surface functionalisation. The unique architecture reported may be applied further to the specific sensing of other gas molecules through deliberate surface modification.

Accordingly, the present invention provides a process for preparing a cross-linked carbon nanotube network, the process comprising:
providing a plurality of reduced carbon nanotubes;
reacting the reduced carbon nanotubes with a conjugated linker precursor to form a covalently cross-linked carbon nanotube network comprising carbon nanotubes covalently linked by a linker formed from the conjugated linker precursor, wherein the linker comprises a conjugated system directly linked to the carbon nanotubes to which it covalently bonds.

Methods of preparing reduced carbon nanotubes are known to the skilled person. For example, methods are described in Clancy, A. J. et al. Charged Carbon Nanomaterials: Redox Chemistries of Fullerenes, Carbon Nanotubes, and Graphenes. Chem. Rev. 118, 7363-7408 (2018).

Reduced carbon nanotubes can be prepared by treating the parent carbon nanotube with a reducing agent.

The reducing agent may comprise a Group I or II metal. Group I metals (alkali metals) can be used to prepare reduced carbon nanotubes. The alkali metal may comprise lithium, sodium, potassium or an alloy thereof. The carbon nanotubes react to form a reduced species with associated counterion. The metal can be introduced as a vapour, molten metal, an amalgam, a eutectic alloy or plasma. Group II metals (alkaline Earth metals may also be used.

Solvated reducing can also be used, i.e. the carbon nanotubes may be treated with a reducing agent in the presence of a solvent. For example, an alkali metal can be dissolved in liquid ammonia for a Birch reduction.

The reducing agent may further comprise a charge transfer agent. The charge transfer agent is an agent which supports electride formation. Charge transfer agents may comprise aromatics. Examples of such charge transfer agents are naphthalene, anthracene, phenanthrene, 4,4′-di-tert-butylbiphenyl, azulene or combinations thereof. Preferably, the charge transfer agent is naphthalene.

A solvent for use in the reduction may preferable be aprotic, for example an ether, an amide or an amine solvent, or a mixture thereof. The ether may comprise alkyl or cycloalkyl ethers. Exemplary ethers include tetrahydrofuran (THF), dioxane, diethyl ether, diisopropyl ether, di-n-butyl ether, di-sec-butyl ether, methyl t-butyl ether, 1,2-dimethoxyethane, 1,2-dimethoxypropane, 1,3-dimethoxypropane, 1,2-diethoxyethane, 1,2-diethoxypropane, 12-crown-4 ether, 15-crown-5 ether, 18-crown-6 ether or combinations thereof. Amine solvents may be used and may comprise tertiary amines. Useful amines may comprise tertiary alkyl or cycloalkyl amines. Exemplary amines include tertiary amine including n-methyl piperidine, n-methyl morpholine, N,N,N′,N′-tetramethyl-1,2-diaminoethane, or combinations thereof. Amide solvents may be used. Exemplary amides include dimethylformamide, N-methyl-2-pyrrolidone, N,N-dimethylacetamide. Amide solvents should preferably be inert towards the alkali metal. The solvent should also be stable in the presence of both the change transfer agent and the reduced carbon nanotubes (formed by treatment with a reducing agent). A preferable amide solvent includes N—N-dimethylacetamide.

Organometallics (e.g., n-BuLi and C₃H₇NHLi) may be used to reduce carbon nanotubes while simultaneously functionalizing with the organic group, allowing the addition of different surface moieties. Thus, the reducing agent may be an organometallic as defined herein, preferably an alkali metal organometallic.

Reduction of the carbon nanotubes can alternatively be performed electrochemically, which has the advantages of simultaneously reducing the carbon nanotube and dissolving the reduced carbon nanotube, if an appropriate solvent (e.g. N,N-dimethylformamide, DMF) is used.

Preferably the reduced carbon nanotube is provided in a solution. Preferably, the solvent is a polar, aprotic solvent as described above. Suitable solvents include N—N-dimethylacetamide, N,N-dimethylformamide (DMF), N-cyclohexyl-2-pyrrolidone (CHP), N-methylpyrrolidone (NMP), dimethylsulfoxide (DMSO) and dimethylacetamide (DMAc). Simultaneous reduction and dissolution of the carbon nanotube, i.e. reductive dissolution, may be advantageous. Reductive dissolution is a favourable method for producing films of cross-linked nanotubes as the electrical properties of the nanotubes are preserved during processing. See FIG. 4(a). Reductive dissolution may achieve high reduced carbon nanotube concentrations whilst retaining their superlative properties.

N—N-dimethylacetamide is a preferable solvent as it is able to stabilise reduced carbon nanotubes, particularly for reductive dissolution.

The term carbon nanotubes according to the present invention refers to nanoscale tubes made substantially of sp² bonded carbon atoms, having a structure based on graphite basal planes that are wrapped or curled to become a tube. Carbon nanotube may be any type of nanotube, that is, it may be any hollow tubular structure having at least one dimension measuring on the nanometre scale.

Carbon nanotubes may be single-walled carbon nanotubes (SWCNTs), double-walled carbon nanotubes or multi-walled carbon nanotubes (MWCNTs) having more than two layers, preferably SWCNTs or double-walled carbon nanotubes. Preferably the carbon nanotubes are SWCNTs and, thus, the reduced carbon nanotube species is nanotubide (SWCNTⁿ⁻).

The carbon nanotubes used in the present invention nanotube may have a smallest inner diameter measuring between about 0.5 nm to about 50 nm, such as about 0.5 nm to about 20 nm, for example between about 0.7 nm to about 10 nm, e.g. between about 0.8 nm to about 2 nm. Small diameter carbon nanotubes are defined herein as carbon nanotubes having diameters of at most about 3 nm, regardless of the number of walls. The nanotube may be of any length. For example, the nanotube may have a length between about 5 nm to about 500 μm, preferably about 10 to about 100,000 nm, preferably about 100 to about 10,000 nm. Preferably, greater than 75 wt % of the nanotubes in the network prepared herein have dimensions in the ranges set out immediately above.

The dimensions of the carbon nanotubes may be determined by microscopic methods (for example atomic force microscopy (AFM) or transmission electron microscopy (TEM) or spectroscopic methods, for example Raman spectroscopy or absorbance/fluorescence spectroscopy. The carbon nanotubes may be dispersed in any suitable way that does not affect the distribution being measured. For example, reductive dissolution may be used. Once dissolved and deposited on a substrate (eg silicon wafer), the length distribution may be established by imaging a sample with AFM and directly measuring the length of the CNTs imaged. Similarly, diameter distribution may be measured by imaging the sample with TEM and determining the diameter of the species present. Diameter can be measured by AFM (using a height measurement), by Raman spectroscopy (via the so-called “breathing mode” frequency), or inferred from absorbance/fluorescence spectroscopy in the visible/nIR range.

The reduced carbon nanotubes are reacted with a conjugated linker precursor to form a covalently cross-linked carbon nanotube network. The carbon nanotube network comprises the carbon nanotubes covalently linked to each other by a linker formed form the conjugated linker precursor. The linker comprises a conjugated system directly linked to the carbon nanotubes. The linker bonds directly to the carbon nanotubes such that no non-conjugated moiety is present between the linker and the carbon nanotubes. Thus, two conjugated systems (the linker and the carbon nanotubes to which in covalently bonds) are directly adjacent.

The conjugated linker precursor comprises at least two functional groups capable of reacting with the wall of the reduced carbon nanotube to form a C—C covalent bond and, hence, a covalently cross-linked network. Preferably, at least two functional groups are located on different atoms of the linker molecule, more preferably at some distance, to maximise the chance of reacting with two different nanotubes.

As would be appreciated, the conjugated linker precursor reacts with the reduced carbon nanotubes to form a linker absent the functional groups. The linker bonds directly via a C—C bond to the carbon nanotubes to form adjacent conjugated systems with the carbon nanotubes to which it covalently bonds. The linker is thus a multivalent moiety.

In a direct linkage, there are no intervening moieties. For example, the conjugated linker precursor reacts with the reduced carbon nanotubes to form a direct C—C linkage where there are no intervening moieties.

The functional groups may be a suitable leaving group enabling the linker precursor to react directly with the sidewalls of the reduced nanotube, for example by an electrophilic substitution reaction, as shown in Scheme 1.

Direct linking of the conjugated linker to the nanotube sidewalls is particularly amenable to creating conjugated networks that aid electrical conductivity. The linker is conjugated such that when it is directly linked to the nanotube the two conjugated systems are adjacent, such that conjugation may be maintained to some extent between adjacent nanotubes.

The conjugated linker precursor (and, hence, the linker) comprises continuous conjugation between each of functional groups. The conjugated linker precursor may comprise non-conjugated substituents, peripheral to the continuous conjugation between each of functional groups. Preferably, the conjugated linker precursor is entirely conjugated between each of functional groups.

The linker is a multivalent (e.g. a divalent) conjugated system, for example comprising one or more aryl or heteroaryl rings, which may be optionally substituted. Said one or more aryl or heteroaryl rings may be bound directly to one another or bound via a trivalent sp² carbon group.

The conjugated linker precursor may be a compound of structure:

X-A′-X

wherein A′ is a divalent conjugated system; and each X is independently a suitable leaving group. X may comprise a halide, sulfoxide, or tosylate. Alternatively, X may be a peroxide or disulphide moiety.

The conjugated linker precursor may be a compound of structure:

X-A-X

wherein A is a divalent conjugated system comprising one or more aryl or heteroaryl rings, wherein A may be optionally substituted; and each X is independently a suitable leaving group. X may comprise a halide, sulfoxide, or tosylate.

A may comprise more than one aryl or heteroaryl rings, each of which may be bound directly to one another or bound via a trivalent sp² carbon group (for example, methine).

A may consist essentially of said one or more aryl or heteroaryl rings and said optional trivalent sp² carbon group and any optional substituent present on said one or more aryl or heteroaryl rings.

A is a conjugated moiety comprising one or more aryl or heteroaryl rings. A may have structure:

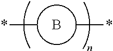

wherein each Ring B is independently an optionally substituted aryl or heteroaryl; n is an integer from 1 to 5; and * indicates the point of attachment to each X.

A may comprise a porphyrin ring. For example, A may have the structure:

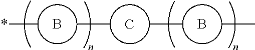

wherein
each Ring B is independently an optionally substituted aryl or heteroaryl;
Ring C is an optionally substituted porphyrin ring;
n is an integer from 1 to 5; and
* indicates the point of attachment to each X.

As will be appreciated, the conjugated linker precursor reacts with the sidewalls of the reduced nanotubes to cross-link adjacent nanotubes, wherein the nanotubes are linked by a conjugated linker having the structure *-A-*, wherein * indicates the point of attachment to each nanotube, i.e. the linker precursor structure, absent the leaving group X.

The linker moiety of the conjugated inker precursor (e.g. group A) may be substituted. It is not essential for such substitution to be conjugated. The substituent can be tailored depending on the target gas when the network is used in gas sensing applications. The substituent can be chosen to tune the binding affinity to the target gas.

For example, amino substituent may increase the binding affinity towards volatile acid gases. Substituents containing carboxylic acid groups may increase binding to amines. Thiourea groups may increase binding of ketones. Noble metal nanoparticles may increase hydrogen sensing. Metallated porphyrin substituents may increase binding towards CO or amines. Calix[4]arenes may be used for the selective detection of aromatic and chlorinated hydrocarbons. Long alkyl chains (for example, C₁-C₂₀alkyl) may be for increased adsorption of aliphatic hydrocarbons. Crown ether groups may be used for enhanced binding of alcohols due to hydrogen binding basicity.

Possible substituents include C₁-C₂₀alkyl, amino (including alkylamino and dialkylamino), carboxylic acid, amino-C₁-C₆alkyl, C₁-C₆alkyl-COOH, and —NHC(S)(NH₂), or a substituent comprising a Noble metal nanoparticle, a porphyrin, a calix[4]arenes or a crown ether.

Substituents that provide additional functional groups capable of reacting with the surface of the reduced carbon nanotube may be used, for example suitable leaving groups.

Group A, comprising one or more aryl or heteroaryl rings, may be optionally substituted. A may be optionally substituted with a substituent as defined herein, including one or more suitable leaving groups (such as halides).

As will be appreciated, where the conjugated linker precursor (e.g. A) is optionally substituted with one or more suitable leaving group (such as halide), the suitable leaving group substituent may act as a functional group capable of reacting with the surface of the reduced carbon nanotube.

The conjugated linker precursor may have more than two functional groups capable of reacting with the surface of the reduced carbon nanotube. For example, the conjugated linker precursor may be a compound of structure:

A-(—X)ₘ

wherein A and X are as defined herein and m is an integer of 2 or more, for example 3 or 4.

A combination of conjugated linker precursors may be used in the process of the invention.

The linker in the cross-linked carbon nanotube network can provide increased sensitivity to target gas analytes, for example when the networks are used in gas sensing applications. Choice of the linker structure, and substitution on the linker, allows tuning of the binding affinity towards different gases.

Altering the linking moiety may significantly affect the resultant film morphology, allowing for tuning of the properties. Further, the crosslinking reaction provides a useful route to incorporating additional functionality into the cross-linked carbon nanotube network.

The cross-linking reaction between the reduced carbon nanotubes and the conjugated linker precursor may be carried out in solution. For example, a polar, aprotic solvent, as used to dissolve the reduced carbon nanotube.

The cross-linking process may be carried out at any reasonable temperature and left for any length of time necessary to complete the reaction, for example so long as the reaction is carried out at a temperature below the boiling point of reaction solvent(s). The reaction is carried out at a temperature of between 10 to 60° C., preferably 15 to 30° C. The reaction time is preferably between 0.1 to 50 hours or more.

During the course of the reaction, the carbon nanotubes are cross-linked to form a gel phase. A “gel” refers to a composition which retains its shape during the drying process and would be known to a skilled person. The gel phase is formed by a continuous network of covalently bound nanotubes within a solvent.

By carrying out the process of covalent cross-linking in a gel phase, the resultant carbon nanotubes can retain their structural integrity during the removal of the solvent. The cross-linking reaction can advantageously be carried out in a saturated solvent environment to avoid solvent evaporation, which may lead to capillary collapse of the fine pore structure and a reduction in available surface area.

The process may further comprise depositing the cross-linked carbon nanotube network on the surface of a substrate.

Reacting the cross-linking reaction may comprise contacting the plurality of reduced carbon-nanotubes with the conjugated linker precursor in a single step.

Alternatively, the cross-linking reaction may comprise two or more steps, where a reduced carbon nanotube is contacted with a conjugated linker precursor and then subsequently contacted with a further reduced carbon nanotube to form a cross-linked carbon nanotube network. The process may comprise a layer-by-layer deposition process, for example comprising the following steps:
providing the substrate;
depositing a reduced carbon nanotube on the surface of the substrate;
contacting the reduced carbon nanotube with a conjugated linker precursor to form a covalently functionalised carbon nanotube;
contacting the covalently functionalised carbon nanotube with a further reduced carbon nanotube to form a covalently cross-linked carbon nanotube network comprising carbon nanotubes covalently linked by a linker formed from the conjugated linker precursor, wherein the linker comprises a conjugated system directly linked to the carbon nanotubes.

Each contacting step may be repeated to build up a three-dimensional cross-linked carbon nanotube network. For example, the process may further comprise:
reacting the cross-linked carbon nanotube network with a further conjugated linker precursor and then reacting the resultant material with a further reduced carbon nanotube.

A layer-by-layer deposition (also described as a molecular layer deposition) process is illustrated in FIG. 4(b) for single-walled carbon nanotubes (SWCNTs). The first SWCNT layer is deposited (for example, by spin-coating) nanotubide onto a substrate (for example, silanised glass). This charged layer is then submerged in a bath of linker precursor, mono-functionalising the available surface until the surface charges are depleted. This forms a covalently functionalised carbon nanotube. Residual leaving group moieties (for example, halides) are available on the covalently functionalised carbon nanotube for grafting to the next SWCNT layer. The yield of mono-grafted to bi-grafted linker is assumed to be high due to the much large concentration of crosslinker molecule and the faster reaction rate of the first grafting. The deposited SWCNTs may advantageously be anchored to the substrate as a result of the covalent interaction with the functionalisation of the substrate (such as an iodopropyl silane layer). This steric restriction may help ensure that the linker precursor does not crosslink SWCNTs within the same layer. Following deposition of the layer of mono-grafted linker, excess reagent may be removed by submersion in a suitable solvent (such as DMAc), before the next layer of SWCNTs is added. The charged nanotubes in solution react with the residual leaving groups remaining from the linker precursor. This reaction forms the second distinct layer of SWCNTs, which is covalently bonded to the previous SWCNT layer by the linker. SWCNTs are deposited until the leaving groups are exhausted, limiting the deposition to a single layer. The cycle is repeated for the addition of each subsequent crosslinked layer, allowing for films with controllable thickness and transparency to be deposited.

By using a molecular layer deposition process, a layer-by-layer architecture may be formed in the cross-linked carbon nanotube network, such that the carbon nanotube junctions (i.e. the cross-linkers) are connected in a series of parallel layers. Molecular layer deposition allows high levels of control over the thickness and transparency of resulting cross-linked carbon nanotube network films, as observed with AFM and UV-Vis spectroscopy. The cross-linked carbon nanotube network may be described as an anisotopic network—orgnaised in layers—unlike any previously known processes. It allows a large number of molecular junctions to be wired in parallel. If these junctions “switch on” in the presence of analyte, a high sensitivity is expected. If you have a “switch off” in the presence of analyte, the additional layers provide sensitivity. The ability to tune the balance of series and parallel molecular junctions (and probe either) is an advantage of this invention.

In an alternative embodiment, the conjugated cross-linker precursor may be deposited on the surface of the substrate and the cross-linked carbon nanotube network may be built up from there.

The cross-linked carbon nanotube network is deposited on the substrate. Deposition can be carried out, for example, by drop-casting or spin-coating. Deposition can be carried out for any time necessary. By carrying out the cross-linking reaction in the presence of the substrate, the cross-linked carbon nanotube network may be deposited as it forms. Thus, deposition may occur concurrently with the cross-linking reaction.

Preferably, the deposition occurs during the cross-linking reaction, such that the cross-linked carbon nanotube is formed on the surface of the substrate. This may be achieved by carrying out the cross-linking reaction in the presence of the substrate. This enables formation of homogenous films.

Advantageously, the layer-by-layer deposition process enables in situ formation of the cross-linked carbon nanotube network on the surface of the substrate forming a homogenous film in a highly controlled manner.

Any suitable substrate may be used. The substrate may be an inert substrate. For example, the substrate may be a glass substrate. The substrate may be a silicon wafer or a non-conductive metal oxide, such as an alumina substrate.

The substrate is preferably a non-conducting substrate.

However, it also possible to use a top to bottom transport measurement, where the aerogel is deposited on a metallic contact (for example a metal substrate or a conducting oxide such as indium tin oxide). Such a conducting substrate may be particularly suitable for the multilayer architecture where a through thickness measurement uses the junctions in the parallel mode intended. In this case, source and drain electrodes are positioned on the top and bottom of the thin film, allowing for through-layer transport to be measured.

The substrate may be functionalised before deposition, for example to aid wetting of the substrate with the cross-linked carbon nanotube network. Functionalisation of the substrate may also aid homogeneity of the deposited cross-linked carbon nanotube network. The substrate may be functionalised with a silane material, i.e. silanised. Suitable silane materials include halo-alkoxysilanes or (3-aminopropyl)triethoxysilane (APTES). Preferably, a silane material is a halo-alkoxysilane, such as 3-iodopropoxysilane or (3-bromopropyl)trimethoxysilane. Alternatively, functionalisation may be carried out with a thiol.

The substrate may be saturated with reduced carbon nanotubes prior to deposition to aid wetting. For example, the process may comprise an initial step of contacting the substrate with reduced carbon nanotubes prior to deposition for a suitable time to ensure adsorption of reduced carbon nanotubes to the substrate, either directly or via functionalisation on the substrate surface.

Alternatively, the substrate may be saturated with the cross-linker precursor. This is particularly advantageous where a layer-by-layer deposition process is used.

The process described herein may be carried out under any suitable conditions. For example, the process may be carried out under inert conditions, for example under argon gas.

The process described herein may be used to form films for a range of potential applications from photovoltaics, sensors and catalysts to conductive inks and coatings.

Also provided herein is a cross-linked carbon nanotube network prepared by the process of the present invention. A cross-linked carbon nanotube network comprises:

a plurality of carbon nanotubes as described herein; and

at least one linker as described herein that covalently links adjacent carbon nanotubes, wherein the linker comprises a conjugated system directly linked to the carbon nanotubes.

The cross-linked carbon nanotube networks provided herein have a high degree of individualisation achieved through the reductive chemistry, which provides a large available surface area for adsorption. This is in contrast to other methods reported that yield networks of bundles. The cross-linking reaction provides a defined open pore structure, allowing for diffusion through the open network. By altering the structure of the linker, the pore structure can be tuned.

Surface area of cross-linked carbon nanotube networks may be measured by the BET method. Ideally, the cross-linked carbon nanotube networks are formed into a bulk monolithic format in order to measure the surface area. The process described herein allows for the provision of bulk monolithic networks in addition to thin film shape. Surface areas may also be measured electrochemically from capacitance measurements.

The cross-linked carbon nanotube network may have an electrical conductivity of about 500 S m⁻¹ or more, preferably about 600 S m⁻¹ or more, preferably about 700 S m⁻¹ or more, preferably about 800 S m⁻¹ or more, preferably about 900 S m⁻¹ or more, preferably about 1000 S m⁻¹ or more. The electrical conductivity may be up to about 1000000 S m⁻¹.

The conductivity of the cross-linked carbon nanotube network may be anisotropic.

For a cross-linked carbon nanotube network prepared by a molecular layer deposition process described herein, the electrical conductivity may be about 10 S m⁻¹ or more.

Electrical conductivity may be determined by measuring the film resistance using a four-point probe. In this method, the film may be contacted by four wires and a current applied to the outer two wires. The voltage drop between the two inner wires is then measured and used to determine a sheet resistance. Electrical conductivity can be calculated from the sheet resistance.

As would be appreciated, greater electrical conductivities can be achieved through the use of longer carbon nanotubes, alignment of CNTs and chemical doping. An important electrical feature of the cross-linked carbon nanotube network films is that there is a molecular element at the inter-carbon nanotube junction, i.e. linker group, modulating the electrical transport upon exposure to different analytes. For sensor application, the modulation may be the key. Higher baseline conductivity may help provide cheaper electronics. The cross-linked carbon nanotube network films described herein provide a high sensitivity and a high selectivity. This may be achieved by having selective binding sites at the junctions aiding the electronic transport through the network

This can be described as wiring together a large number of molecular junctions. In molecular electronics, molecular junctions are attractive but measurements difficult. Described herein is practical method for measuring a large number of molecular junctions.

The physical properties of the cross-linked carbon nanotube networks described herein may be distinct over other thin films in the degree of individualisation of the carbon species and the fine filament structure. Pronounced van Hove bands (distinct peaks/bumps) in the UV-Vis indicate that the majority of the carbon nanotubes remain as individual species and small bundles in the solid state. This is a direct result of the reductive dissolution and crosslinking process described herein. Scanning electron microscopy provides further evidence for the fine carbon nanotube scaffold structure, with few large bundles or agglomerates observed. The high level of individualisation and fine pore structure indicates a large active surface area, properties ideally suited to gas sensing applications.

Advantageously, the cross-linked carbon nanotube networks are formed using a molecular layer deposition process, such that the carbon nanotubes are connected in a series of parallel layers. This may be determined by comparing in-plane conductivity to through-thickness conductivity. In-plane conductivity may be measured by making both contacts on the same surface of a film, whereas through-thickness measurements require one contact on the top surface of the film and the other contact on the bottom surface. An in-plane conductivity that is significantly larger than the through-thickness conductivity will indicate that the carbon nanotubes are arranged in distinct layers with alternating layers of crosslinker. It is predicted that the through-thickness conductivity will decrease with each subsequent layer, whilst the in-plane conductivity increases.

After deposition of the cross-linked carbon nanotube network, the solvent may be removed, for example the network may be dried.

The removal of solvent may comprise a first step of solvent exchange, for example with at least one solvent having lower surface tension than the initial solvent. The term “surface tension,” as used herein, refers to the attractive force in any liquid exerted by the molecules below the surface upon those at the surface/air interface, which force tends to restrain a liquid from flowing. The term “low surface tension,” as used herein may refer to liquids having a surface tension of less than or equal to about 30 mN/m as measured at 25° C. and atmospheric pressure. After solvent exchange, the carbon nanotube network may be dried.

Some network may be dried without solvent exchange and others will need very low surface tensions. Whether a particular network requires such solvent exchange will depend on the individual properties of the network, i.e. the gel. The lower density, higher surface area networks have more desirable properties but tend to be less robust so may need solvent exchange or other controlled drying technique familiar to the skilled person. The properties of the network will depend on the dimensions of the carbon nanotubes and the cross-link density.

The cross-linked carbon nanotube network according to the present invention is preferably an aerogel. As used herein, the term “aerogel” refers to a highly porous material of low density, which is prepared by forming a gel and then removing liquid from the gel while substantially retaining the gel structure. Preferably, an “aerogel” according to the present invention comprises a carbon nanotube network wherein the volume change on drying of the gel is less than about 30%, preferably less than about 20%, preferably less than about 10%, preferably less than about 5%. Aerogels have open-celled microporous or mesoporous structures. Typically, they may have pore sizes of less than about 1000 nm and surface areas of greater than about 100 m² per gram. Preferably they may have pore sizes of less than about 200 nm and surface areas of greater than about 400 m² per gram. They may often have low densities, e.g., from about 1000 mg/cm³ down to as little as about 1 mg/cm³ preferably in the range of about 15 to 500 mg/cm³. Exceptionally, unlike other existing aerogels, those produced from carbon nanotubes, may have low densities, high surface areas, but large pore sizes.

Pore size may be measured by mercury porosimetry, which is suitable for larger macro pores (diameter>50 nm). For smaller mesopores (about 2-50 nm) and micropores (<2 nm) nitrogen porosimetry may be used. Applying Brunauer-Emmett-Teller (BET) theory to the adsorption-desorption isotherm allows the specific surface area to be determined. The pore size distribution is then calculated from the experimental isotherm using further analysis such as the Barrett-Joyner-Halenda (BJH) method or density functional theory (DFT).

Preferably, aerogels are materials in which the liquid has been removed from the gel under supercritical conditions.

Drying the deposited network may be carried out by supercritical drying or lyophilisation, preferably to form an aerogel. The most common method for supercritical drying involves the removal of the solvent with supercritical carbon dioxide, and this may be used in the present invention. Critical point drying removes solvent without crossing the liquid-gas phase boundary, potentially avoiding forces that may apply force to the network fine structures and cause rebundling of the carbon nanotubes.

Drying the deposited network may be carried out at room temperature and/or ambient pressure. The network may be dried by inert gas flow (such as N₂ flow), particularly where the network was formed by a layer-by-layer deposition process described herein.

This process may be a more versatile procedure to fabricate an aerogel since it does not require supercritical CO₂, or a freezing-vacuum process. The aerogel can be obtained by simply drying the gel. The objective is to evaporate the solvent producing the minimum volume reduction when obtaining the aerogel from the gel.

Aerogels prepared according to the present invention allow the gel to be cast in predetermined shapes. The idea is to control the final shape by controlling the shape in the gel phase. The present process also allows for the formation of a large gel to form a large aerogel.

It is desirable that the resultant carbon nanotube networks contain as few impurities as possible. Such impurities include residual reagents, surfactants, additives, polymer binders and the like. The presence of these impurities can lead to an increase in the density of the carbon nanotube networks as well as reducing the electrical conductivity and surface area of the carbon nanotube aerogel. Impurities may be removed during the solvent exchange process. Since the process according to the present invention does not require the use of a substantial amount of such additives or reagents which are often hard to remove, carbon nanotube networks with high electrical conductivity, large surface area and low density can be obtained. The total amount of impurities present in the carbon nanotube network may be less than 5 wt. %, and even more preferably less than 1 wt, preferably measured after the solvent has been removed.

Preferably, each carbon nanotube used in the present invention has high electric conductivity and allows a current flow at a current density of greater than 10 MA/cm², preferably greater than 100 MA/cm² or more. A network of carbon nanotubes is therefore thought to display excellent electrical conductivity and current density, compared to existing carbon aerogels.

In addition, carbon nanotubes have desirable intrinsic mechanical characteristics, including high strength, stiffness, and flexibility, at low density. These properties make carbon nanotubes desirable for many industrial applications, and lend desirable properties to the resulting aerogel networks.

The shape of the aerogel or xerogel can be controlled by controlling the shape of the vessel used during the gelation step. The density of the final aerogel can be controlled by varying the volume fraction of nanotubes within the initial gel.

The cross-linked carbon nanotube network may be provided (for example, deposited) on a substrate as described herein. The cross-linked carbon nanotube network may be provided as a film on said substrate.

Also provided herein is a film comprising the cross-linked carbon nanotube network. The film may have a thickness of about 1 nm to about 10 μm, for example, about 1 to about 500 nm.

Film thickness may be measured by scoring the film to create a scar or step. The height difference between the substrate and the film surface can be accurately determined by AFM for very thin films (<1 um) or with a surface profiler for thicker films. Alternatively, electron microscopy can be used to measure the thickness of the film cross section.

Also provided herein is layer-by-layer deposition process (a molecular layer deposition process) for forming a cross-linked nanotube network, wherein the process comprises:
depositing a reduced carbon nanotube on a surface of a substrate as described herein;
contacting the reduced carbon nanotube with a linker precursor to form a covalently functionalised carbon nanotube;
contacting the covalently functionalised carbon nanotube with a further reduced carbon nanotube to form a covalently cross-linked carbon nanotube network comprising carbon nanotubes covalently linked by a linker formed from the linker precursor.

Each contacting step may be repeated to build up a three-dimensional cross-linked carbon nanotube network. For example, the process may further comprise:
reacting the cross-linked carbon nanotube network with a further linker precursor and then reacting the resultant material with a further reduced carbon nanotube.

The linker is a multivalent (e.g. divalent) moiety comprising at least two functional groups capable of reacting with the wall of the reduced carbon nanotube to form a C—C covalent bond and, hence, a covalently cross-linked network.

Preferably, the linker is a rigid linker. A rigid linker is sufficiently rigid to as to avoid ‘backbiting’, whereby by leaving groups react at the same carbon nanotube, as shown in Scheme 2, below.

As would be appreciated, the rigid linker must be sufficiently rigid to avoid the leaving groups appearing on the same side of the rigid linker. As used herein, therefore, “rigid” means the linker molecule conformational restrictions and steric hindrance would favour binding to two carbon nanotubes, rather than forming two bonds to a single carbon nanotube. Preferably, at least two functional groups are located on different atoms of the linker molecule, more preferably at some distance, to maximise the chance of reacting with two different nanotubes.

It will be appreciated that the rigid linker precursor may be a conjugated linker precursor such that the process is according to a process described above, wherein the covalently cross-linked carbon nanotube network comprises carbon nanotubes covalently linked by a conjugated linker formed from the conjugated linker precursor. However, the process does not need to be limited to a conjugated linker precursor.

The rigid linker precursor may be a compound of structure:

X—Y—X

wherein Y is a multivalent rigid system; and each X is independently a suitable leaving group as described herein.

Rigidity for the linker may be provided by one or more cycloalkyl or aryl groups.

Y may comprise a cycloalkyl group. Y may consist essentially of a cycloalkyl group. Exemplary cycloalkyl groups include cyclohexyl and adamantane. Y may comprise (preferably consist essentially of) a polyoctahedral silsequioxane

Y may be a group of structure

*—Y′—CH₂—Y′—*

wherein each Y′ is independently a C₆-C₁₀ monocyclic or bicyclic aryl ring or a 5-10 membered monocyclic or bicyclic heteroaryl ring and * indicates the point of attachment to each X. Each Y may independently be phenyl or thiophenyl.

The rigid linker precursor may be 1,4-dihalocyclohexane, for example, 1,4-diiodocyclohexane. The rigid linker precursor may be bis(4-bromophenyl)methane or 5,5-dibromo-2,2-dithienylmethane.

A combination of rigid linker precursors may be used in the process of the invention.

As will be appreciated, the rigid linker precursor reacts with the sidewalls of the reduced nanotubes to cross-link adjacent nanotubes, wherein the nanotubes are linked by a rigid linker having the structure *—Y—*, wherein * indicates the point of attachment to each nanotube, i.e. the rigid linker precursor structure, absent the leaving group X.

The rigid linker moiety of the rigid inker precursor (e.g. group Y) may be substituted as described herein. As will be appreciated, where the rigid linker precursor (e.g. Y) is optionally substituted with one or more suitable leaving group (such as halide), the suitable leaving group substituent may act as a functional group capable of reacting with the surface of the reduced carbon nanotube.

The rigid linker precursor may have more than two functional groups capable of reacting with the surface of the reduced carbon nanotube. For example, the rigid linker precursor may be a compound of structure:

Y—(—X)ₘ

wherein Y, X and m are as defined herein.

It is not essential that the linker precursor is a rigid linker. Full backbiting of the linker may be avoided by saturating the surface of the carbon nanotube with linker precursor to form a form a saturated covalently functionalised carbon nanotube. Thus, any linker precursor may be used as long as it comprises at least two functional groups capable of reacting with the wall of the reduced carbon nanotube to form a C—C covalent bond.

Accordingly, each step of contacting the reduced carbon nanotube with a linker precursor to form a covalently functionalised carbon nanotube may comprise contacting the reduced carbon nanotube with a linker precursor for a sufficient time such that the carbon nanotube is saturated with the covalent functionalisation of the linker, i.e. such that all available sites on the nanotube surface, subject to steric requirements, are covalently functionalised, leaving reactive functional groups on the grafted linker molecules exposed. As would be appreciated, the extent to which the carbon nanotube will be saturated will depend on the steric constraints of the linker molecule.

The process may be carried out under any suitable conditions as described above, for example inert conditions.

The process may be used to form films for a range of potential applications from photovoltaics, sensors and catalysts to conductive inks and coatings as described above.

Also provided herein is a cross-linked carbon nanotube network prepared by this process. A cross-linked carbon nanotube network comprises:
a plurality of carbon nanotubes as described herein; and
at least one linker as described herein that covalently links adjacent carbon nanotubes. The cross-linked carbon nanotube network is formed using reduced carbon nanotubes and a layer-by-layer approach so shares many of the advantages in terms of porosity, homogeneity and conductance as discussed above.

Gas Sensing Applications

The cross-linked carbon nanotube networks and films described herein may be used in gas sensing applications. Accordingly, the cross-linked carbon nanotube network and film described herein may be used as a gas sensor or for sensing gas.

Also provided herein is a gas sensor comprising the carbon nanotube network or film described herein. A gas sensor described herein (for example, a gas sensor for sensing a target gas) may comprise:
first and second electrodes;
a carbon nanotube network as described herein (preferably as a film);
wherein the first and second electrodes are in electrical contact with the carbon nanotube network. The sensor may be in the form of a sensor array.

In use, when the gas sensor or sensor array is exposed to the target gas, the target gas is bound by carbon nanotube network. This changes the impedance of the carbon nanotube network between the first and second electrodes. This change in impedance can be measured and used to determine the concentration or presence of the target gas. Thus the carbon nanotube network of the gas sensor or sensor array may be a chemiresistor.

The carbon nanotube network of the gas sensor or sensor array may be a transistor. For example, the sensor or sensor array may be a chemical field effect transistor (ChemFET). The cross-linked carbon nanotube network may provide the conducting channel between source and drain electrodes in the transistor. In use, a voltage may be applied to a third ‘gate’ electrode, allowing for modulation of the conductance in the source-drain channel. The channel current is measured upon exposure to a gas, at a range of gate voltages, allowing for accurate determination of the concentration or presence of the target gas with high sensitivity.

Any suitable electrode material may be used. For example, each electrode may independently be a metal electrode, such as gold, silver, nickel or platinum, and palladium some commonly used. Gold deposited on chromium may be used as the electrode material. In general, inert, high work function metals are preferred. The junction between the metal and the carbon nanotube network may contribute to sensing activity.

The cross-linked carbon nanotube network may be provided on a substrate as described herein. The substrate may be inert.

As would be appreciated, the substrate may act as an electrode in the sensor or sensor array and, thus, may be formed from any suitable electrode material.

For a transistor, the substrate may act as a “back gate” and, thus, may be formed from a suitable electrode material. Preferably, however, the gate electrode is provided in addition to a substrate.

The gas sensor or sensor array may further comprise a voltage source, wherein the voltage source is configured to apply an electrical potential between the first and second electrodes.

The gas sensor or sensor array described herein may be combined with machine learning to provide a fingerprint of gases that are present in a sample, for example in the air.

Also provided herein is a process for sensing a target gas comprising:
exposing a gas sensor or sensor array as described herein to a gas sample comprising the target gas, such that the target gas is bound by the carbon nanotube network;
measuring impedance of the carbon nanotube network layer by applying an electrical potential to the carbon nanotube network; and
determining the concentration of the target gas based on the impedance of the carbon nanotube network layer.

The concentration of the target gas may be measured in parts-per notation, for example parts per million, as a partial pressure, a percentage weight, a percentage volume, a number of moles per unit volume or a number of moles per unit mass.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

### Definitions

As used herein, alkyl refers to a straight or branched hydrocarbon chain. An alkyl may have from 1 to 10 carbon atoms optionally 1 to 6 carbon atoms. Exemplary alkyl groups include, without limitation, methyl, ethyl, n-propyl, i-propyl, n-butyl, i-butyl, s-butyl, t-butyl, 2-methylbutyl, n-pentyl, s-pentyl, n-hexyl, 2-ethylhexyl, n-heptyl, n-octyl, etc. An alkyl may be unsubstituted or substituted with one or more substituents as defined herein.

As used herein, aryl refers to an aromatic hydrocarbon ring. As used herein, heteroaryl refers to an aryl ring containing one or more heteroatoms (for example 0, S or N). Aryl and heteroaryl groups may be mononuclear, i.e. having only one aromatic ring (for example, phenyl or phenylene), or polynuclear, i.e. having two or more aromatic rings which may be fused (for example, napthyl or naphthylene), individually covalently linked (for example, biphenyl), and/or a combination of both fused and individually linked aromatic rings. Aryl groups may contain from 6 to 20 carbon atoms, or from 6 to 12 carbon atoms. An aryl may be fused to one or more aryl or cycloalkyl rings to form a polycyclic ring system. Exemplary aryl groups include, without limitation, phenyl, biphenylene, triphenylene, [1,1′:3′,1″]terphenyl-2′-ylene, naphthalene, anthracene, binaphthylene, phenanthrene, pyrene, dihydropyrene, chrysene, perylene, tetracene, pentacene, benzpyrene, fluorene, indene, indenofluorene, spirobifluorene, etc. Preferably aryl is phenyl. Exemplary heteroaryl groups include, without limitation, pyrrolyl, furanyl, thiophenyl, imidazolyl, oxazolyl, thiazolyl, triazolyl, pyrazolyl, pyridinyl, pyrazinyl, pyridazinyl and pyrimidinyl, etc. Preferably heteroaryl is thiophenyl or pyrrolyl. An aryl or heteroaryl may be unsubstituted or substituted with one or more substituents as defined herein.

As used herein, cycloalkyl refers to a cyclic alkyl group. A cycloalkyl may have from 3 to 20 cyclic carbon atoms, from 3 to 15 carbon atoms, or from 3 to 10 carbon atoms. Cycloalkyl includes bridged, fused and/or spiro ring systems, such as decalin, norbornane and spiro[5.4]decane. Exemplary cycloalkyl groups include cyclopropyl, cyclobutyl, cyclopentyl, cyclooctyl, decalin, norbornane and spiro[5.4]decane etc. A cycloalkyl may be unsubstituted or substituted with one or more substituents as defined herein.

As used herein, a halide may be selected from the group consisting of —F, —Cl, —Br, —I.

As used herein, a porphyrin is a group comprising four pyrrole rings linked through the α-positions by four methine groups to form an aromatic macrocyclic structure.

Reference is now made to the following examples, which illustrate the invention in a non-limiting fashion.

Consumer Products

The consumer products of the present invention may include air fresheners; air freshener devices; air cleaning devices; air filtration devices; oral care products, including tooth brushes and toothpaste; mouthwashes; personal care products including body wash, shampoo, conditioner, antiperspirant and deodorant; absorbent articles including diapers and adult incontinence articles; feminine care products; razors; dish detergent; fabric care products including detergents, softeners, dryer sheets, scent boosters; cleaning implements including dusters and mops; surface cleaners; cleaning wipes; paper products including paper towel and tissue, and the like.

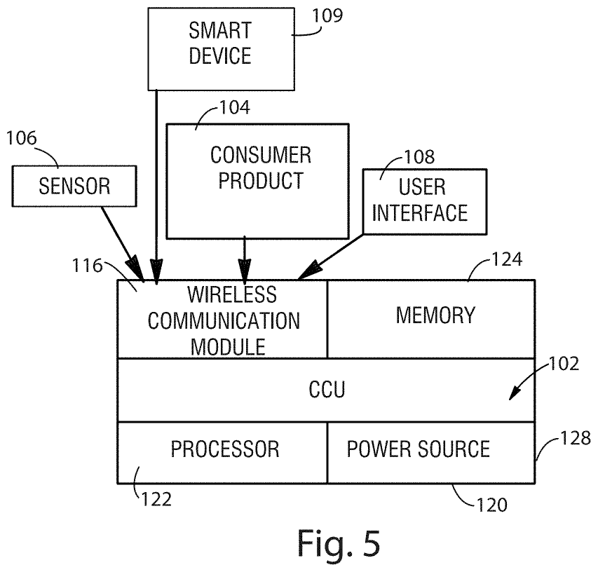

The consumer product may be a smart consumer product, device, or both that is wirelessly connected with a controller and/or the sensor. The consumer product may include a wireless communication module in order to be connected and automated through the system and included in the methods of the present disclosure. The consumer products may be communicably connectable with the CCU. With reference to FIG. 5, the consumer products may include a wireless communication module 136 in order to communicate with the CCU and/or consumer product composition dispenser 104. A smart consumer product may be configured as a tooth brush, a razor, an absorbent article, a feminine care product, a cleaning implement, an air freshener device or an air cleaning device.

The sensor may be physically disposed on the consumer product or the sensor may be an separate element of the system that is movable independent of the consumer product. Placing the sensor directly on the consumer product may allow readings in very close proximity to the consumer product no matter where the consumer product is positioned at any given time and allows for the sensor and the consumer product to be moved simultaneously.

The consumer product 104 may require user intervention to respond to data generated by the sensor 106. For example, the sensor 106 may send an incoming signal to the CCU. The incoming signal from the CCU may read a high or low level of a target gas concentration in the space surrounding the sensor, as compared with a setpoint for the target gas concentration. The CCU may alert the user, causing the user to respond by selecting and using a particular consumer product that is capable of mitigating or boosting the concentration a particular target gas.

The consumer product 104 may be in the form of a consumer product composition dispenser. The consumer product composition dispenser may be used for the delivery of a consumer product composition to the atmosphere or onto an inanimate surface. Such consumer product composition dispenser may be configured in a variety of ways. For example, the consumer product composition dispenser may be configured as an energized dispenser (i.e. powered by electricity; or chemical reactions, such as catalyst fuel systems; or solar powered; or the like). Exemplary energized consumer product composition dispensers include a powered delivery assistance means which may include a heating element, a piezo element, thermal ink jet element, fan assembly, cold air diffusion with an air pump to aerosolize, automated sprayer, automated aerosol, hydrogen fuel cell, or the like. More particularly, the consumer product composition dispenser may be an electrical wall-plug consumer product composition dispenser, a non-limiting example of an electrical wall-plug consumer product composition dispenser is described in U.S. Pat. No. 7,223,361; a battery (including rechargeable battery) powered consumer product composition dispenser having a heating and/or fan element. In energized devices, the consumer product composition dispenser may be placed next to the powered delivery assistance means to diffuse the volatile material. The volatile material may be formulated to optimally diffuse with the delivery assistance means. An exemplary consumer product composition dispenser includes an air freshening dispenser.

The consumer product composition dispenser may be configured as a non-energized dispenser. An exemplary non-energized consumer product composition dispenser includes a reservoir and, optionally, capillary, wicking means, or an emanating surface, to help volatile materials passively diffuse into the air (i.e. without an energized means). A more specific example of a consumer product composition dispenser includes a delivery engine having a liquid reservoir for containing a volatile material and a microporous membrane enclosing the liquid reservoir as disclosed in U.S. Pat. Nos. 8,709,337 and 8,931,711.

The consumer product composition dispenser 104 may also be configured for use as an aerosol sprayer or a non-aerosol sprayer. The consumer product composition dispenser 104 can be programmed to automatically deliver a consumer product composition to the atmosphere. The sprayer may be manually operated by a user or may be automatically operated through electromechanical means.

An air cleaning device may utilize ionization, filtration, plasma, UV light (e.g. UVC, UVA, UVB) catalytic coatings, metal oxide coatings, and/or hydroxyl radical technology for use in individual rooms or small spaces (e.g. bedrooms, bathrooms, automobiles, etc.), and/or whole house central air conditioning/heating systems (e.g. HVAC). In the case of air cleaning to disinfect the air, the air cleaning device may dispense particles, such as vaporized or aerosolized droplets of hydrogen peroxide, glycols, triethylene glycol, hydroxyl radicals, hypochlorous acid, essential oils, ozone, quaternary amines, positive and/or negative charged ions, or other actives to disinfect or sanitize the air. The air cleaning device can also be a ventilation device in the bathroom or kitchen areas that is desired to evacuate the air in that room (i.e. a bathroom exhaust or a kitchen hood over the stove to ventilate the air).

The consumer product, including a consumer product composition dispenser and/or air cleaning device, may also be configured as a mobile robot that goes to where the sensor is reading one or more target gases outside of the setpoint and may use one or emore available technologies to boost or mitigate the concentration of the target gas in the location of the sensor. For example, the mobile robot may utilize air freshening, air cleaning, and/or air sanitizing technologies in the location where the sensor is detecting the high or low level of the target gas.

The system may include one or more consumer products. The system may include an array of consumer products that may be movable to different rooms within a housing or building. Moreover, a house or building may include one or more consumer product composition dispensers that are positioned in the same room or in different rooms.

The freshening composition dispenser may be able to contain and keep separated more than one consumer product composition, including at least two different consumer product compositions, or at least two different consumer product compositions, or at least three consumer product compositions.

The consumer composition may be an air freshening composition, including a perfume composition and/or a malodor control composition. A consumer composition may include an insect repellant. A consumer composition may include a biocide.

The consumer product composition may comprise volatile materials. Exemplary volatile materials include perfume materials, volatile dyes, materials that function as insecticides, essential oils or materials that acts to condition, modify, or otherwise modify the environment (e.g. to assist with sleep, wake, respiratory health, and like conditions), deodorants or malodor control compositions (e.g. odor neutralizing materials such as reactive aldehydes (as disclosed in U.S. 2005/0124512), odor blocking materials, odor masking materials, or sensory modifying materials such as ionones (also disclosed in U.S. 2005/0124512)).

The consumer product composition may include a perfume mixture of perfume raw materials to provide a desirable scent in the air. The consumer product composition includes a mixture of volatile aldehydes that are designed to deliver genuine malodor neutralization (and not function merely by covering up or masking odors). A genuine malodor neutralization provides a sensory and analytically measurable (e.g. gas chromatograph) malodor reduction. Thus, if the consumer product composition delivers genuine malodor neutralization, the consumer product composition will reduce malodors in the vapor and/or liquid phase. The consumer product composition may comprise a mixture of volatile aldehydes that neutralize malodors in vapor and/or liquid phase via chemical reactions. Such volatile aldehydes are also called reactive aldehydes. Volatile aldehydes may react with amine-based odors, following the path of Schiff-base formation. Volatiles aldehydes may also react with sulfur-based odors, forming thiol acetals, hemi thiolacetals, and thiol esters in vapor and/or liquid phase.

The consumer product composition may include various other ingredients, including, but not limited to: surfactants; acid catalysts; polymers; buffering agents; solubilizers; antimicrobial compounds; preservatives; wetting agents; aqueous carrier; diluents; the like; and combinations thereof.

Central Communications Unit

The CCU 102 can be configured in various different ways. The CCU 102 may be configured to receive incoming signals from the one or more components of the system 100 and send outgoing instructions to one or more components of the system 100, for example the consumer product(s) and/or smart devices in the system.

With reference to FIG. 5, the CCU 102 may be communicably connectable with various components of the system 100, including the sensor(s) 106, user interface(s) 108, the consumer product 104 using a wireless communication link 107. Various wireless communication links may be used, including 802.11 (Wi-Fi), 802.15.4 (ZigBee, 6LoWPAN, Thread, JennetIP), Bluetooth, combinations thereof, and the like. Connection may be through an ad hoc Mesh Network protocol. The CCU 102 may include a wireless communication module 116 in order to establish a wireless communication link 107 with the CCU 102 with various components of the system. Any module known in the art for establishing the communication links can be utilized.

The CCU 102 may comprise a processor 122. The processor 122 may be configured and programmed to carry out and/or cause to be carried out the one or more advantageous functionalities of the system 100 described herein. The processor 122 may be physically disposed within a CCU 102 or may be remotely located on a computer, special computer, smart device such as a phone or tablet, server, intranet, border router, cloud-based system, the like, or combinations thereof. The processor 122 can carry out algorithms stored in local memory; special-purpose processors or application-specific integrated circuits; algorithms carried out or governed remotely by central servers, or cloud-based systems, such as by virtue of running a Java virtual machine that executes instructions provided from a cloud server using Asynchronous JavaScript and XML or similar protocols. The algorithms may be regularly repeated, such as on a particular time interval. The algorithm may repeat hourly, daily, weekly, etc and may be configured to operate on different schedules at different times of the day or different days of the week. A different algorithm may be used depending on whether the user is present or not present in the space.

The CCU 102 may comprise a memory 124. The memory may be configured to store set points; incoming signals, such as sensor measurements and status indicators; algorithms; and the like. The memory may be a local memory within the CCU 102 such as a flash drive, hard drive, read only memory, or random access memory. Or, the memory may be configured as a remote memory on a computer, smart device such as a phone or tablet, on a server, or on a cloud-based system. The memory 124 can be accessible to the processor 122 in a variety of ways.

The processor and/or the memory of the CCU 102 may be disposed within a housing of the CCU 102. The CCU 102 may be connected with or separate from various components of the system 100. For example, the CCU 102 may be physically connected with the consumer product. The CCU 102 may be permanently positioned in a building in a separate room or location from other components such as the smart device and/or the consumer product 104, for example. The CCU 102 may be configured as a smart thermostat. A smart phone or tablet may be used as the CCU. The CCU may be communicably connectable with a computer, smart phone, or tablet.

The CCU may include a clock or may wirelessly communicate with a clock. The CCU may be communicably connectable with a clock on a computer, smart device, or on the internet.

FIG. 5 illustrates an exemplary CCU 102 having the processor 122 and the memory 124 disposed within a housing 128. The CCU 102 shown in FIG. 5 may be disposed on or within a consumer product 104 or smart device 109. While FIG. 5 illustrates a processor 122 and a memory 124 disposed within the housing 128, it is to be appreciated that the processor 122 and/or the memory 124 may be remotely located relative to the CCU 102.

Incoming signals may pass through a CCU unit comprising a transmitter that transmits the incoming signals to the remote memory. Incoming signals may also be directly received by a component that is wirelessly communicating with the component sending the signals.

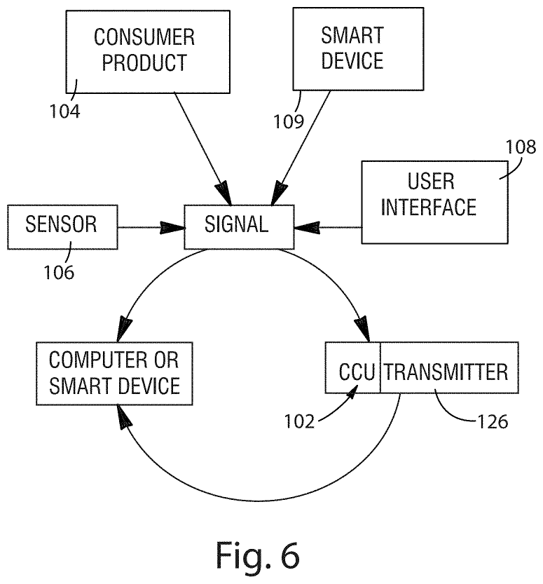

FIG. 6 illustrates multiple exemplary flows of incoming signals from various components of the system 100 to a remote memory. The incoming signals may flow directly from the sensor 106, consumer product 104, smart device 109, or various other components to a computer or smart device through a wireless communication link or through a transmitter of the CCU to a remote memory. The processor 122 may access the incoming signals from the memory 124. The processor 122 may access the memory 124 through a wired or wireless communication link.

The processor 122 may be configured to compare incoming signals to set points stored in the memory 124. The processor is able to retrieve stored set points from the memory 124 to compre. The system may include a user feedback loop to assist the user in selecting appropriate setpoints. For example, a user may not appreciate what the appropriate levels of particular target gases should be. The user interface or CCU may allow the user to answer questions or select from different prompts on the user's desired interests. Exemplary questions or prompts may relate to the user's preference on scent intensity, identification and quantification of sensitivities that the user may have to particular smells, times of day or activities where the user may desire a particular scent in the air or increased cleaning of the air. Based on the responses from the user, the CCU may select specific setpoints or may alter the setpoints, such as through machine learning. The user feedback loop allows a user to modify setpoints, and subsequent operation of the consumer products may be adjusted in response to the setpoint modifications from the user.

The consumer feedback loop may occur before the start of operation to help with algorithm selection and/or during operation to check that the setpoints are still meeting the consumer's needs or interests.

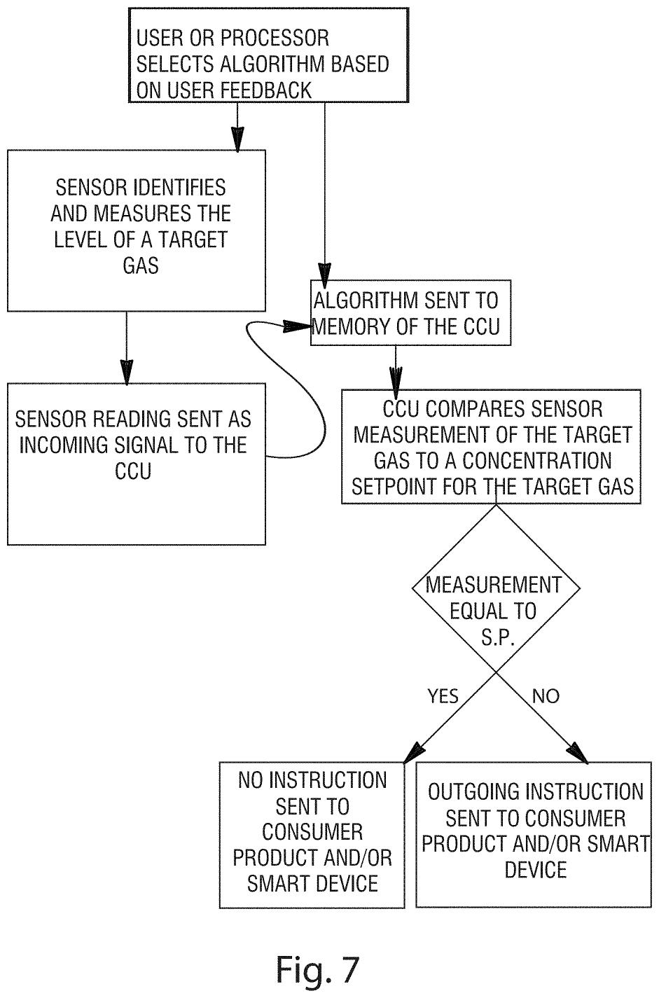

As discussed above, the CCU may be configured to run various algorithms that operate one or more consumer products 104 in response to the incoming signals from the sensor(s) 106. FIG. 7 illustrates an exemplary algorithm that may be used by the CCU 102 to control the consumer product(s) 104 and/or a smart device(s) 109 based on incoming signals from the sensor(s) 106. The sensor may be configured to one or more target gases of interest. If the sensor detects a target gas, the sensor 106 may identify the target gas(es) and concentration(s) of the target gas(es) and send those details as incoming signals to the CCU.

With continuing reference to FIG. 7, in an exemplary algorithm that is either selected by the user or selected by the processor based on feedback from the user, the processor 122 may be configured to compare incoming signals from the sensor 106 on a particular target gas to set points for the target gas that are stored in the memory 124. If the value of the incoming signal to the CCU 102 is different from the target gas concentration or concentration range set point stored in memory 124, the processor 122 sends an outgoing instruction to a particular consumer product 104 or smart device 109 that is identified by the CCU as being able to mitigate or boost the level of the particular target gas. The CCU may send an outgoing instruction to a selected consumer product 104 to either turn the consumer product 104 ON or OFF, depending on the desired outcome and the utility of the specific consumer product 104. If the value of the incoming signal from the sensor 106 is equal to or substantially equal to the set point concentration for that specific target gas, the processor 122 will then either send an outgoing instruction to the consumer product or smart device to either turn ON or OFF or will send an outgoing instruction to the consumer product 104 to change the operational settings of the consumer product, such as the flow rate, intensity, or the like. An algorithm such as described may be repeated on set time schedule, such as hourly, daily, weekly, etc and may be configured to operate on different schedules at different times of the day or different days of the week, and/or may operate differently when the user is present or not present within the space.

“Substantially equal to” may include an acceptable tolerance between the set point and the concentration measured by the sensor that may be programmed into an algorithm. As such, sensor concentration measurements that are substantially equal to the set point may be treated as being equal to the set point by the processor.

The memory 124 may be configured to store multiple set points. For example, there may be different set points for different times, time periods of a day and there may be different set points for different days of the week. The CCU may use a clock to determine which set point is to be used for a particular time of day and/or day of the week.

The setpoint may be a desirable or acceptable concentration or concentration range for a specific target gas of interest. The memory may store setpoints for one or more target gases of interest and one or more sensors may be used to simultaneously identify and measure multiple target gases of interest.

The algorithm may be programmed to send outgoing signals to the sensor to take sensor measurements at specific times during the day, or at set intervals throughout the day.

The CCU may be configured to use sensor measurements from different sensors located within a house or building. Different sensors may be used at different times of the day and/or different days of the week or different sensors may simultaneously take measurements.

Set points may be used to control when and for how long the consumer product(s) are turned ON. The duration of time that a specific consumer product may be turned ON may be in the range of about 5 minutes to about 60 minutes, or in the range of about 10 minutes to about 30 minutes. For example, the set point may be configured as a predetermined duration of time after the consumer product is turned ON so that the consumer product may be turned off at a preprogrammed duration of time.

Upon receiving an incoming signal from the sensor that a target gas is outside of the setpoint, the CCU may be configured to provide suggested actions to the user. For example, the CCU may recognize the types of consumer products connected with the system or the user may instruct the CCU of the list of consumer products that the user may has at their disposal. Based on that list of consumer products, the CCU can alert the user of the particular consumer product or products that may be helpful in mitigating or boosting the particular target gas or gases. The CCU can either automatically send an outgoing instruction to one or more wirelessly connected consumer products and/or may send a notification or instruction to the user about suggested manual actions by the user with one or more identified consumer products. The CCU may also send an outgoing instruction to the user to open a window or increase the ventilation in a home.

The CCU may be configured as a thermostat such as the thermostat shown in FIG. 2. The thermostat may include a processor or memory, or the thermostat may communicate with a remote processor and/or memory. The thermostat may include a user interface. The thermostat may be a NEST® learning thermostat, a LUTRON® thermostat etc. The processor may compute optimal set points from an algorithm based on user preferences of target gas concentrations.

A machine learning algorithm can learn a user(s) preferred set points at various times of day and/or days of the week and/or can be used to program a more energy efficient algorithm, for example. An exemplary learning system is used in a NEST® learning thermostat. An exemplary learning system is also described in U.S. Pat. No. 9,115,908. The processor then transmits the optimal set points to the memory which then stores the set points. The machine learning algorithm may be used to make adjustments and calibrations for drift and background levels of sensor measurements through user feedback and/or calibration to increase sensitivity and selectivity of target gases.

Devices in the system, including consumer products, sensors, and smart devices, may interact with each other through the CCU such that events detected by one device may influence actions of another device or the current status of one device may influence actions of another device.

User Interface

The systems and methods of the present disclosure may include one or more user interfaces 108. The user interface 108 may be configured in various different forms. A user can interact with the user interface 108 to adjust set points as well as connect the sensors 106 through the CCU 102 for viewing of live sensor data on the user interface. The CCU 102 could also connect to the internet or intranet and pass through information, such as sensor measurements and the set points to a server for the purpose of remote monitoring on a user interface 108. The user interface may be integral with the CCU or may be separate. One or more user interfaces may be connected with the system.

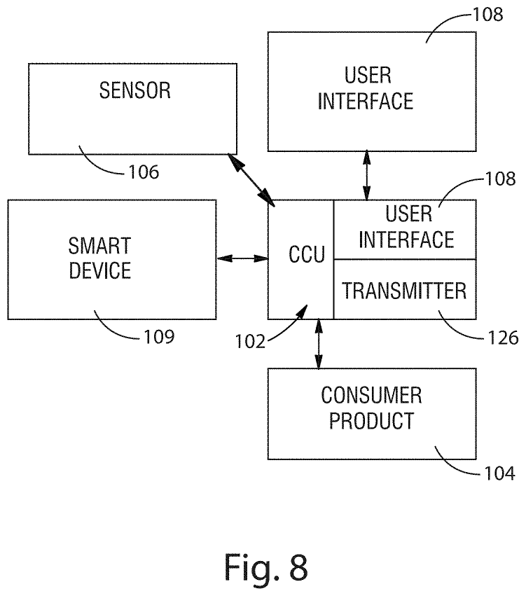

FIG. 8 illustrates an exemplary system having more than one user interface. In FIG. 8, a first user interface is connected with the CCU and a second user interface is a remote user interface. The remote user interface may be in the form of a computer or handheld smart device.

Where the CCU is configured as a thermostat, the thermostat may include a user interface where the user can adjust temperature set points by pushing buttons or turning dials, for example.

The user interface may be configured as a program, HTML website, or a Native application that is accessible by a user through a computer or handheld smart device. A handheld smart device may include an iPhone®, iPad®, or an Android® or Microsoft® based system. The user interface may be accessible on a computer such as a desktop, laptop, or tablet.

The CCU, the sensor, and/or the user interface may include an indicator light or series of indicator lights to alert the user to one or more of the sensors measures a target gas concentration outside of the setpoint. The user may respond to the indicator light by selecting and using a consumer product to mitigate or boost the target gas or gases.

The system of the present disclosure may include a handheld smart device or computer that comprises the CCU 102, including the processor 122, memory 124, and/or user interface 108.

### EXAMPLES

Materials and Methods

Purified HiPco was purchased from Nanointegris and degassed for 10 h at 500° C. under high vacuum (10⁻⁶ mbar), before introduction into the glovebox. Sodium (99.95% ingot), DMAc (anhydrous 99.8%), naphthalene (99%) p-diiodobenzene (99%), and 2,5-dibromoaniline (97%) were purchased from Sigma Aldrich Ltd. (UK). Ethanol (96%) and acetone (99.8%) were purchased from VWR Ltd. (USA). Dry air (O₂/N₂ 20/80 v/v) was purchased from BOC. Reductive dissolutions and aerogel film deposition were performed under inert conditions in a glovebox (mBraun, O₂<0.1 ppm, H₂O<0.1 ppm).

Measurements

TGA was performed using a METTLER Toledo TGA-DSC 1 under a N₂ atmosphere. Samples were held at 100° C. for 30 min under N₂ flow of 60 mL/min, then ramped at 10° C./min to 800° C. XPS spectrometer equipped with a MXR3 Al Kα monochromated X-ray source (hv=1486.6 eV). X-ray gun power was set to 72 W (6 mA and 12 kV). Charge compensation was achieved using the FG03 flood gun using a combination of low energy electrons and the ion flood source. Argon etching of the samples was done using the standard EX06 Argon ion source using 500 V accelerating voltage and 1 μA ion gun current. Survey scans were acquired using 200 eV pass energy, 1 eV step size and 100 ms (50 ms×2 scans) dwell times. All high-resolution spectra (C1s, and O1s) were acquired using 20 eV pass energy, 0.1 eV step size and 1 second (50 ms×20 scans=1000 ms) dwell times. Samples were prepared by pressing the sample onto double side sticky carbon based tape. Pressure during the measurement of XPS spectra was <1×10-8 mbar. Thermo Avantage software was used for data interpretation. Casa XPS software (version 2.3.16) was used to process the data. The quantification analysis was carried out after subtracting the baseline using the Shirley or two point linear background type. Peaks were fitted using GL(30) lineshapes; a combination of Gaussian (70%) and Lorentzian (30%). All XPS spectra were charge corrected by referencing the fitted contribution of C—C graphitic like carbon in the C1s signal 284.5 eV.

Raman spectroscopy was performed with a Renishaw InVia Raman Spectrometer and a 532 nm laser, with a 1800 line/mm grating. Statistical D/G measurements were performed by comparing the relative intensities of the D band and G band, from multiple spectra comprising a map of an area of >100 μm×100 μm. Scanning electron microscopy (SEM) was performed on a Zeiss Gemini Sigma300 at an accelerating voltage of 10 keV. Samples were adhered to aluminium stubs with carbon tabs and the film contacted with silver paint. Atomic force microscopy (AFM) were performed with dynamic mode on a hpAFM with AFM controller (NanoMagnetics Instruments, UK) using Nanosensor tapping mode probes. Micrographs were processed with NMI Image Analyzer (v1.4, NanoMagnetics Instruments), utilising the in-built height and line correction functions and the scar removal function.

### Example 1—Cross-Linking and Deposition

Reductive Dissolution and Crosslinking

In a typical synthesis, a bulk solution was prepared by adding equimolar quantities of sodium ingot (30 mg, 1.30 mmol) and naphthalene (167 mg) to DMAc (15.0 mL), giving a sodium concentration of 2 mg_{Na} mL_{DMAc}⁻¹. The dark green solution was stirred for 18 h. Sodium naphthalide solution (2.24 mL, 2.24 mmol_{Na}) was then added to degassed SWCNT powder (40 mg) to give a sodium:carbon stoichiometry of 1:10. The solution was then diluted with additional DMAc to yield a concentration of 1.8 mgsw_{CNTs} mL_{DMAc}⁻¹ and optimum [Na] of 15 mM. The solution was stirred overnight with a glass stir bar to yield a homogenous solution before use.

Conjugated Linker Precursor Synthesis

BocDBA synthesised according to Fracaroli, A. M. et al. Metal-organic frameworks with precisely designed interior for carbon dioxide capture in the presence of water. J. Am. Chem. Soc. 136, 8863-8866 (2014). The procedure was adapted from Fracaroli et al. Briefly, tetrahydrofuran (THF) (20 mL) was degassed by sparging with nitrogen, before the addition of 1,5-dibromoaniline (2.00 g, 8.0 mmol), 4-dimethylaminopyridine (98 mg, 0.8 mmol) and di-tbutyl decarbonate (5.2 g, 24 mmol). The reaction mixture was stirred for 5 h at 40° C. The THF was removed in vacuo, before addition of MeCN (80 mL) and LiBr (2.14 g, 24.6 mmol). The suspension was stirred for 18 h at 65° C., before cooling to room temperature and evaporating to dryness. The crude product was dissolved in DCM and purified by flash silica column chromatography, eluting with hexane:AcOEt 20:1, to yield the product as a pale yellow solid (2.81 g, 35%). ¹H NMR (400 MHz, CDCl₃), [ppm]: 1.54 (s, 9H), 6.99 (br s, 1H), 7.02 (dd, J=8.5, 2.4 Hz, 1H), 7.34 (d, J=8.5 Hz, 1H), 8.40 (d, J=2.4 Hz, 1H)

Aerogel Film Deposition

Glass microscope slides were cut into squares and submerged in piranha solution (25:75 H₂O₂(30%):H₂SO₄(94%)) for 20 min before rinsing 3 times with HPLC water. The substrates were dried under vacuum before drop casting excess 3-iodopropyl-trimethoxysilane onto the top surface. After 30 min the residual silane was rinsed off with DMAc before drying under vacuum.

Aerogel Film: Carbon nanotubide (1.8 mgsw_{CNTs} mL_{DMAc}⁻¹) (150 μL) was added to a vial and equimolar quantities (crosslinker:Na) of crosslinking agent added in solution to the nanotubide. 12 μL of this gelling nanotubide was then promptly dropcast onto pre-silanised glass substrates, contained in a DMAc saturated atmosphere, for 24 h. The films were then subjected to a series of solvent exchanges (MeOH, MeOH/HCl (0.1M), Isopropyl alcohol/H₂O), followed by acetone for 24 h, before supercritical drying with liquid CO₂. FIG. 9(a).

UV-Vis spectroscopy of the resultant semi-transparent thin film exhibits pronounced Van Hove singularity peaks (FIG. 9(c)), that correspond to M₁₁ and S₂₂ transitions. These absorption bands reveal the presence of individualised SWCNTs and small-diameter bundles, that are retained in the solid state following critical point drying. The film is low density with a measured thickness of 240 nm (FIG. 9(d,e)) whilst retaining 35% transparency.

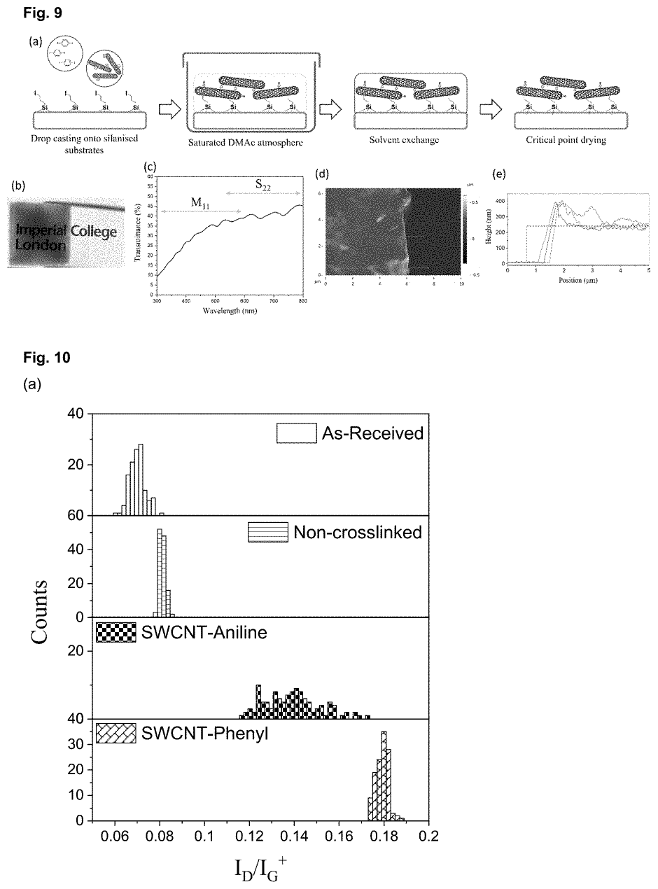

Cross-linked Carbon Nanotube Network Functionalisation and Characterisation Aerogel films were produced using p-diiodobenzene (DIB) to provide a network without a functional binding site, and a Boc-protected 2,5-dibromoaniline (BocDBA) to provide an amine binding site. Statistical Raman spectroscopy (SRS) shows an increase in the relative intensity of the defect-induced mode (˜1350 cm⁻¹) for both functionalised films when compared to both a non-crosslinked film and the pristine SWCNT material, indicating covalent grafting to the SWCNTs. Higher intensity defect modes are observed for DIB than BocDBA which is attributed to the higher reactivity of aryl iodides with nanotubide. In each case, the D band increase is relatively small, in particular for the non-crosslinked aerogel film, showing that the reductive dissolution route is generally non-destructive to the sp² lattice. X-ray photoelectron spectroscopy (XPS) reveals small amounts residual iodine and bromine in the p-DIB and BocDBA crosslinked films respectively, indicating mono-grafted species. The aniline crosslinked network contained 0.84% of nitrogen atoms, giving an estimate of 1 grafted unit per 90 SWCNT carbon atoms. Small increases in oxygen content for each film indicate that oxygen functional groups are added during the aerogel film synthesis, with the oxygen discharging step the likely contributor. Further evidence for crosslinker grafting is provided with thermogravimetric analysis (TGA) of crosslinked networks produced in a monolithic format (FIG. 10(b)). Larger mass loss of 19% and 14% is observed for phenyl- and aniline crosslinked networks respectively, reinforcing the evidence from TGA that p-DIB grafts in higher yield than BocDBA. Only a modest mass loss is observed for the non-crosslinked control sample, showing that the reductive dissolution in isolation does not introduce many additional functional groups.

Microstructure

X-Ray Photoelectron Spectroscopy (XPS)

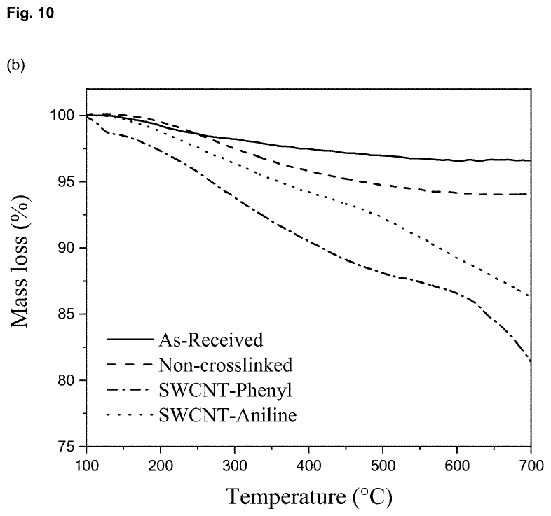

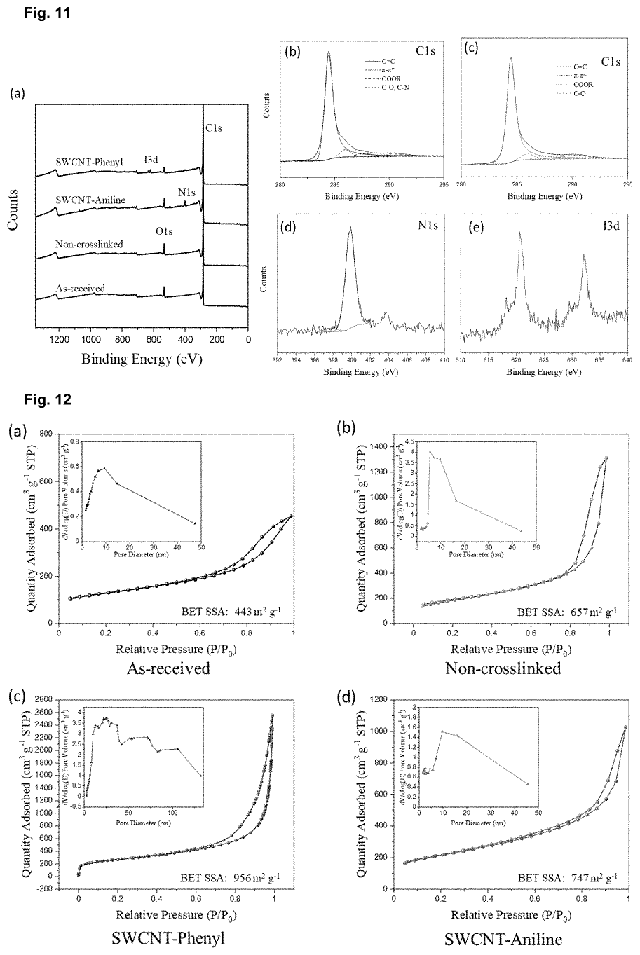

XPS of the SWCNT samples contain large C1s peaks (285 eV), smaller O1s peaks (532 eV) and further peaks that correspond to surface functionalisation (FIG. 11). The C1s scans reveal the samples to be comprised mostly of sp2 hybridised carbon, as is expected of SWCNT films. The aniline connected network contained a strong N1 s peak at 400 eV, equating to 1.8 at. (%) of nitrogen atoms, giving an estimate of 1 grafted crosslinker per 53 SWCNT carbon atoms, and confirming the presence of the binding site in the sample. XPS of the crosslinked films reveals small amounts of residual iodine and bromine in the p-DIB and BocDBA functionalised films, respectively, indicating a small number of mono-grafted species present in the sample.

N2 Isotherms/BET

N2 porosimetry revealed the crosslinked aerogel bulk monoliths to be mesoporous materials with a characteristic type IV isotherm, that is distinguished by the adsorption-desorption hysteresis (FIG. 12). The HiPco starting material exhibited BET specific surface area (SSA) of 443 m2 g-1, which is significantly small than the theoretical limit of 1315 m2 g-1 for closed SWCNTs, reflecting the highly bundled state of the material observed through SEM. SSA of 657 m2 g-1 was recorded for non-crosslinked bulk monolith, a substantial increase in active surface over the raw SWCNTs, emphasising the efficacy of the reductive dissolution route in exfoliating the bundles and retaining the open network. The aniline crosslinked network had a BET specific surface area (SSA) of 747 m2 g-1, whilst the phenyl crosslinked aerogel exhibited larger SSA of 956 m2 g-1. Both crosslinked networks displayed larger active surface than the non-crosslinked aerogel, reiterating the importance of the crosslinking step in retaining the open microstructure. The larger SSA of the phenyl network when compared to the aniline network reflects the higher crosslinking yield observed. The added structural links secure the SWCNT framework in a defined position, preventing collapse and improving SSA, which reinforces the observations made with SEM about the altered microstructure. The pore size distribution of crosslinked aerogels, again shows the networks to contain predominantly mesopores.

N₂ sorption isotherms of crosslinked aerogel monoliths, revealed a mesoporous material with specific surface area (SSA) of 956±2 m² g⁻¹ for the phenyl crosslinked network (FIG. 12(c)). This illustrates an increase over the non-crosslinked control, due to the presence of structural crosslinks securing the SWCNT framework in a defined position. The difference observed between the two crosslinked samples is attributed to a greater number of crosslinks grafted to the SWCNT sidewalls. At high magnification, scanning electron microscopy (SEM) of the aerogel thin films reveals a highly interconnected random network of small diameter bundles (FIG. 13(c-e)). In both crosslinked cases, a fine uniform pore structure is observed, whilst in absence of a crosslinker, a coarser network is seen with a greater degree of rebundling apparent, which is also reflected in the SSA. With an average pore diameter of around 5 nm for crosslinked networks, high surface area is achieved, whilst maintaining sufficient spacing for efficient gas diffusion throughout the whole structure. A summary of the characterisation data for each crosslinked network, as well as the non-crosslinked control and the de-gassed starting material is provided in Table 1.

TABLE 1
Characterisation data for de-gassed HiPco and reductively synthesised aerogels.

| | Linker | Raman | XPS at. (%) | | | | | Wt. loss at | BET Specific |
|---|---|---|---|---|---|---|---|---|---|
| | Reagent | D/G Ratio | C1s | O1s | N1s | Br3d | 13d | 700° C. (%) | Surface area (m²g⁻¹) |
| As-received | — | 0.066 | 94.4 | 5.6 | — | — | — | 3 | 443 |
| Non-crosslinked | — | 0.079 | 95.4 | 4.6 | 0.2 | — | — | 6 | 657 |
| SWCNT-Aniline | 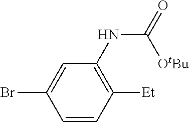 | 0.136 | 94.8 | 3.3 | 1.8 | 0.1 | — | 14 | 747 |
| SWCNT-Phenyl | 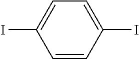 | 0.179 | 96.5 | 2.4 | 0.6 | — | 0.2 | 19 | 956 |

### Example 2—Layer-by-Layer Deposition Method

Materials and Methods

Purified HiPco were purchased from Nanointegris and degassed for 10 h at 500° C. under high vacuum (10⁻⁶ mbar), before introduction into the glovebox. Sodium (99.95% ingot), DMAc (anhydrous 99.8%), naphthalene (99%) and p-diiodobenzene (99%) were purchased from Sigma Aldrich Ltd. (UK). Isopropanol and deionised water was purchased from VWR Ltd. (USA). Dry air (O₂/N₂ 20/80 v/v) was purchased from BOC. Reductive dissolutions and molecular nanolayer depositions were performed under inert conditions in a glovebox (mBraun, O₂<0.1 ppm, H₂O<0.1 ppm). All reactions were performed at room temperature and multilayer films used for Raman spectroscopy were deposited on silicon wafer.

Reductive Dissolution

A bulk ‘nanotubide’ solution was prepared by adding equimolar quantities of sodium ingot (30 mg, 1.30 mmol) and naphthalene (167 mg) to DMAc (15.0 mL), giving a sodium concentration of 2 mg_{Na} mL_{DMAc}⁻¹. The dark green solution was stirred for 18 h. Sodium naphthalide solution (2.24 mL, 2.24 mmol_{Na}) was then added to degassed SWCNT powder (40 mg) to give a sodium:carbon stoichiometry of 1:10. The solution was then diluted with additional DMAc to yield a concentration of 1.8 mgsw_{CNTs} mL_{DMAc}⁻¹ and optimum [Na] of 15 mM. The solution was stirred overnight with a glass stir bar to yield a homogenous solution before use.

Molecular Nanolayer Deposition

Reduced SWCNTs were diluted with further to DMAc to give a mass loading of 0.2 mg mL⁻¹. The first layer was deposited by spincoating 200 μL of the diluted SWCNT⁻ onto glass substrates that had been silanised with 3-iodopropyltrimethoxysilane. The substrate was then submerged in a 15 mg mL⁻¹ solution of crosslinker p-DIB for 10 min, followed by submersion in neat DMAc for a further 5 min. Submersion of the substrate in a bath of nanotubide for 10 min, followed by neat DMAc again for 5 min, completed a single crosslinking cycle. The 4 individual submersions were repeated for each subsequent layer of SWCNTs and crosslinker. Multilayer films were then removed from the glovebox and residual charge quenched with dry oxygen gas (20:80 O₂:N₂ v:v). Finally, films were washed with deionised water and isopropyl alcohol, before drying under nitrogen flow.

Microstructure

Helium ion microscopy of multilayer films reveals a porous network of highly individualised SWCNT species and small bundles. The large degree of individualisation reflects the reductive exfoliation route which is highly effective at separating larger bundles. FIG. 14a shows the initial layer of SWCNTs, and in most regions, the film appears to consist of a either single nanotubes or very small number of stacked SWCNTs. Following the addition of a second layer, in absence of a crosslinker (FIG. 14b), there appears to be additional SWCNTs lying on top of the first layer, although the absolute coverage with nanotubes is largely unchanged. Conversely, the crosslinked film (FIG. 14c) shows a greater addition of SWCNTs to the second layer and a much larger density of carbon material. The quantity of material deposited is increased when using a crosslinking layer, indicating that the covalent grafting is controlling the deposition, and is required to anchor a substantial quantity of SWCNTs to the film.

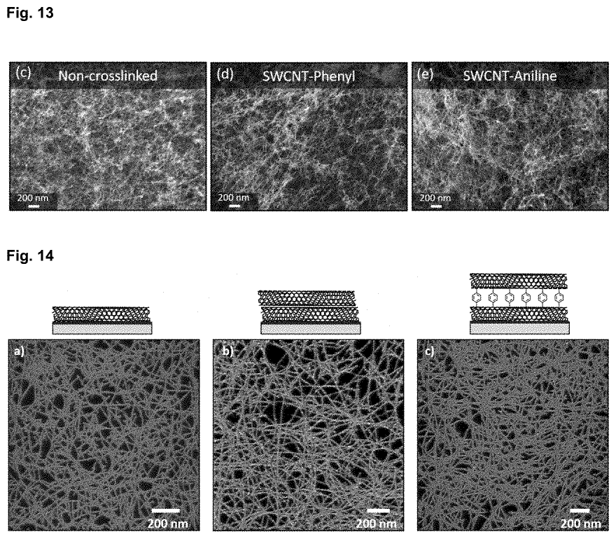

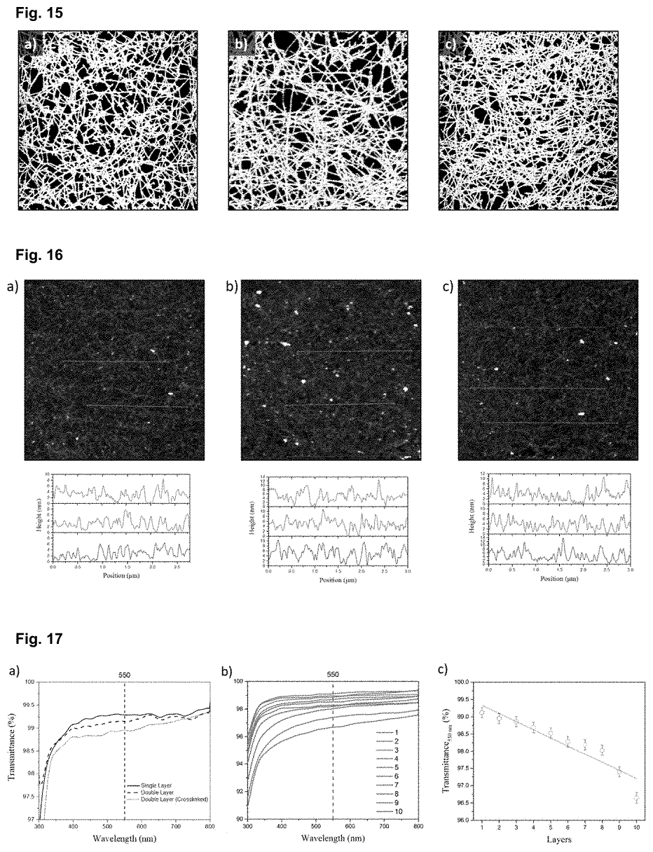

By thresholding the micrographs into black and white pixels (FIG. 15), a value can be determined for the total coverage of SWCNT compared to substrate background, giving an indication as to the amount of SWCNTs present in the second layer. SWCNT coverage (C_{CWCNT}) of the single layer was determined to be 1.35, whilst only marginally higher coverage of 1.44 was calculated when a second layer was added without crosslinker. The crosslinked film yielded C_{SWCNT} of 1.81, showing that much more material is deposited when there is the iodo-group available for grafting to the incoming layer.

Atomic force microscopy (AFM) was utilised to determine the thickness of the initial SWCNT layer and then the thickness of subsequent layers (FIG. 16). As with HIM, the AFM reveals highly interconnected networks of individual SWCNTs and small diameter bundles. The first layer, deposited via spincoating, had a thickness of 4-6 nm, with a roughness of 1.6 nm. Upon addition of the 2^{nd} layer, the thickness increased to 6-9 nm for both crosslinked and non-crosslinked films, although a larger roughness of 2.9 nm was observed in absence of crosslinking layer compared to 2.1 nm for the crosslinked film. It is hypothesised that is a result of the grafting reaction with the crosslinker, pulling the SWCNTs into the plane of the film, reducing the roughness. Whilst the non-crosslinked film has more material sitting out of plane, increasing the roughness. A thickness increase of 2-3 nm upon the addition of the second layer, suggests that a monolayer of SWCNTs is added, before the carbon-halide bonds are depleted and the reaction terminated.

UV-Vis Spectroscopy

To reinforce the argument that the crosslinking reaction is controlling the deposition, UV-visible spectroscopy was performed (FIG. 17). Pronounced Van Hove singularity bands are observed in the films, which again reflects the high degree of individualisation that is observed through microscopy. The presence of these discrete energy levels is typically observed in solution, however their presence in the solid state shows that the molecular layer deposition precludes significant rebundling. The transmittance of SWCNT films at 550 nm gives a good indication to the amount of material present in the film. A larger reduction in transmittance is observed for the crosslinked double layer, than for the non-crosslinked film, reaffirming the role of the crosslinking in controlling the deposition. A linear drop in transmittance is observed for each subsequent layer, up to a total of 10 layers, demonstrating the degree of control that can be attained over film thickness and transparency (FIG. 17b,c).

Raman Spectroscopy

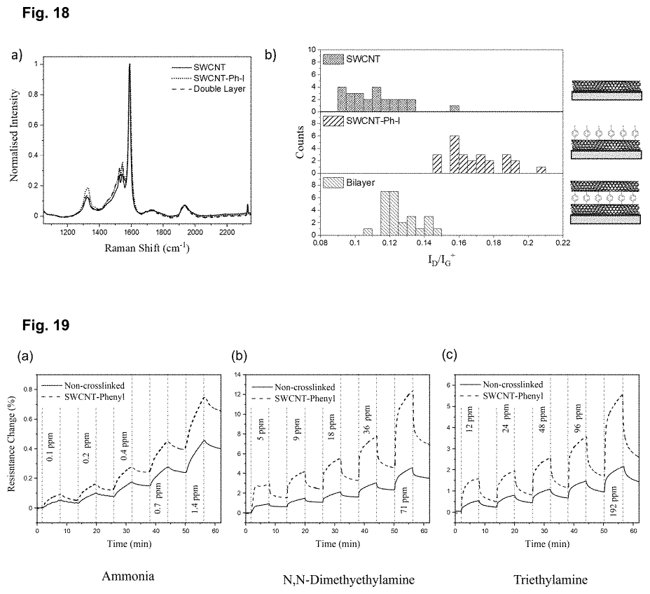

The nature of the MND was probed further through Raman spectroscopy, a technique that measures vibrational modes, allowing for the determination of the types of bonds present in a structure. The defect-induced mode, appearing at 1300 cm⁻¹, is an indicator of damage to the sp² carbon lattice, and the ratio of this peak to the graphitic peak (I_{D}/I_{G}⁺), appearing around 1600 cm⁻¹, is used to determine the degree of sidewall functionalisation. Statistical Raman spectroscopy confirms functionalisation of the first SWCNT layer with crosslinking reagent, as the average I_{D}/I_{G}⁺ shifts from 0.11±0.015 to 0.17±0.016 (FIG. 18b). The crosslinked bilayer exhibits a smaller D-band than the mono-functionalised SWCNT layer, showing that additional SWCNT material has been deposited. The I_{D}/I_{G}⁺ of 0.13±0.010 is larger than observed for the single layer, indicating reaction with the residual halide groups of the first layer

Reported herein, is a method for the layer-by-layer molecular nanolayer deposition (MND) of highly interconnected crosslinked SWCNT films that exploits the reductive crosslinking chemistry. Assembling films layer upon layer provides a route to hierarchical structures with highly tuneable cross-functionalities such as electrical conductivity, optical transmittance and thickness. Reductive dissolution yields solutions of highly individualised species, that can be assembled into multilayer films through crosslinking with dielectrophiles. A combination of AFM, HIM, UV-Vis and Raman spectroscopy have been employed to characterise the deposition process, revealing that covalent reaction with the crosslinker drives the deposition of SWCNTs. The layer-by-layer approach affords very tight control over the film properties, with thickness and transparency dependent on the number of layers. Moreover, room temperature reaction conditions and a simple bath dip-coating process facilitate potential scale-up of the deposition. The method demonstrated may be applied to thin films for a range of potential applications from photovoltaics, sensors and catalysts to conductive inks and coatings.

### Example 3—Gas Sensing

Gas Sensing Measurements

SWCNT samples were placed in a custom-made flow cell and the resistance recorded (Keysight4410A DMM) using a 4-wire measurement whilst exposed to various gas analytes. Gas mixtures were generated from permeation tubes and diffusion tubes with Nitrogen carrier gas in a re-conditioned KinTek H₂O Span Pac.

Sensors were zeroed by heating to 100° C. under nitrogen flow. Measurements were taken with sequential 6-minute exposures of analyte followed by 6 minutes of pure carrier gas. Analyte concentrations were adjusted by controlling the temperature of the permeation device and the dilution gas flow rate.

Analytical Response to Amines

Aerogel films were fashioned into the active component of chemiresistor gas sensors by contacting the SWCNT surface with source and drain contact probe. Gas analytes of varying concentration, ranging from low parts-per-billion (ppb), to parts-per-million (ppm), were delivered to the sensor in dry nitrogen carrier gas. Phenyl-crosslinked networks exhibited high sensitivity to the odorous amines, ammonia, n,n-dimethylethylamine (DMEA) and triethyl amine (TEA) (FIG. 19). Ammonia was detected at concentrations as low as 0.1 ppm for both non-crosslinked and phenyl-crosslinked networks. Both DMEA and TMEA elicit strong responses from 55 ppm down to 4.5 ppm and 32 ppm down to 2 ppm respectively. The phenyl crosslink does not contain functional groups that would contribute significantly to analyte binding and so signal transduction is caused by adsorption of amines to the SWCNT surface. The increasing resistance trend is explained by the electronic structure of SWCNTs, with surface adsorption of amines causing a partial charge transfer of electrons to the SWCNT network. This reduces the availability of holes, the majority charge carrier in the p-type network, limiting electrical conductance. Very high sensitivity is observed, which can be attributed to the reductively exfoliated and subsequently crosslinked SWCNTs, providing a large surface for amine adsorption.

Non-crosslinked networks still exhibit good performance, given they have still undergone the same individualisation process, however the device sensitivity is still measurably lower than in the case where a crosslinker has been used to prevent rebundling. Another reason for sensitivity enhancement of more individualised sensor networks may be the larger number of CNT junctions. In highly conductive networks, conductivity is limited at the tube-tube junction, so perturbing the electronic structure at these loci will yield a more significant response. A greater degree of rebundling will cause intra-tube sensing responses to be the dominant effect, while in the more exfoliated crosslinked networks, the inter-tube effect is more prevalent.

Sensing Volatile Acids

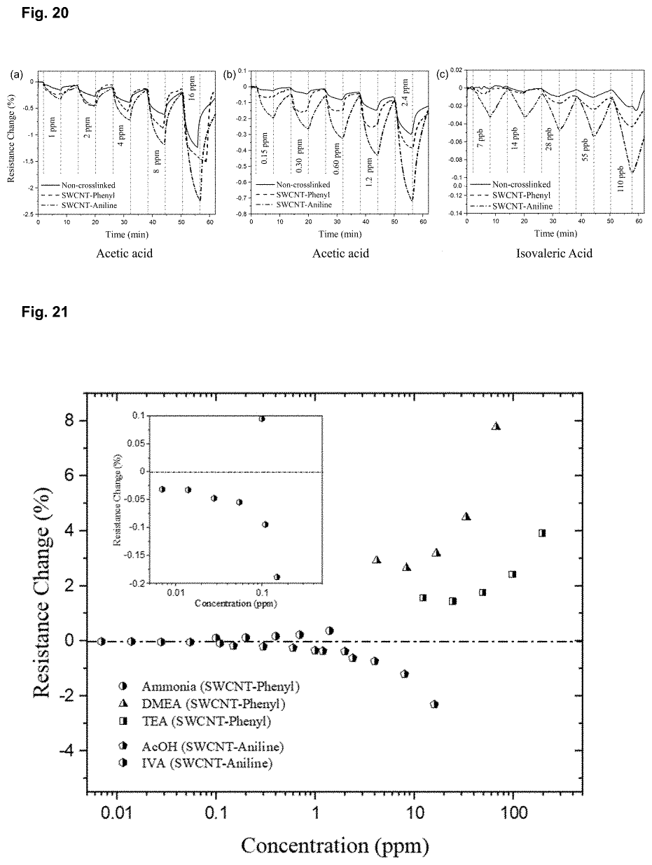

Further investigation was undertaken into the gas sensing behaviour of aerogel films with the VOCs, acetic acid and isovaleric acid. The networks crosslinked with an aniline bridge were utilised, both in attempt to increase the binding affinity for acid molecules using the free amine sites, and to localise binding events to the tube junctions, increasing the dominance of the inter-tube sensing mechanism. A Boc-protected aniline was used in the synthesis, to favour grafting between the C—Br and the charged SWCNT surface, precluding unwanted side reactions at the nitrogen containing group. Free amines were detected with XPS (Table 1), indicating that the selective binding site is present in the sample. Upon exposure to acetic acid, a strong signal was observed, with a significant linear resistance change seen both from 1 ppm up to 16 ppm (FIG. 20(a)) and over a lower concentration range of 0.15-2.4 ppm. The increase in conductivity contrasts with the effect seen upon exposure to amine molecules favouring the explanation that charge transfer from the SWCNT to the adsorbed electron-withdrawing acid occurs. The reduction in electron density increases hole conductivity in the network, eliciting a response in the form of lower resistance. A large increase in sensitivity is observed when compared to aerogel films that do not contain the aniline crosslinks. This increase indicates that there is additional adsorption of acid onto the network due to the complimentary interaction between the free amine binding site and adsorbed acid. Additionally, where acetic acids molecules are interacting at amine sites, there will be charge transfer from the conjugated network, adding to the doping effect, giving a larger sensing response. By positioning the analyte binding sites at the inter-tube junction, a single binding event may modulate transport along a whole percolation pathway. It is hypothesised that binding of this type elicits a larger response than adsorption at a sidewall position where electrical transport is not limited. While networks that do not contain the selector moiety are still sensitive to acetic acid in the low ppm range, the sensitivity is diminished by a factor of 2-3, emphasising the key role the amine functional group plays in the response.

Similar behaviour was observed with isovaleric acid, another low molecular weight carboxylic acid, with a measurable drop in resistance seen as low as 7 ppb, and linear response covering the dynamic range up to 110 ppb. Control networks, lacking the aniline linker, did not show a significant resistance change until exposure to 28 ppb, with the % resistance change smaller than aniline-crosslinked samples.

Aniline-crosslinked aerogel film sensors were reproducible and stable for periods longer than 6 months. Similar behaviour was observed for both acid analytes, indicating the interfacing between the molecule and SWCNT network is the same, pointing to a charge transfer from the carboxyl group. By extrapolating the dynamic range for acetic acid responses, the detection limit appears to be in the same region as isovaleric acid. The detection limit reaches that of the human olfactory system, 7 ppb, which has evolved over millennia to be extremely sensitive to such harmful malodourous compounds. By enhancing sensitivity towards acid molecules, a degree of selectivity has been imparted onto the sensing material.

The full dynamic range for both types of aerogel thin film sensors displays sensitivity to both electron rich and electron poor analytes over a vast concentration range from low ppb up to ppm (FIG. 19). It shows a linear relationship between analyte concentration and measured resistance response for each analyte measured, allowing for quantification of gases in real samples. The inverse trends seen for acid and amine analytes are indicative of a charge transfer mechanism.

Aerogel film workfunctions were determined by ambient pressure photoemission spectroscopy (APS), whereby surface electrons are liberated by the photo electric effect. The energy of the onset of photoemission provides a workfunction energy.

In summary, a unique aerogel thin film deposition has been demonstrated, whereby a solution of reduced carbon nanotubes (SWCNTs) is crosslinked into a 3-dimensional network. SWCNT aerogel films exhibit a high degree of individualisation with a uniform mesoporous structure. These observed properties in the active material, resulted in chemical sensors that are sensitive in the ppb regime to a range of acid and amine gases. Functionalisation with free-amine binding sites enhanced sensitivity to acid compounds, imparting a degree of selectivity towards this class of molecules. A shift in the Raman G peak position and the opposite change in resistance for the two classes of compounds, indicates that signal transduction is dependent on chemical doping from adsorbed analyte molecules. By altering the crosslinking motif, this network architecture may be applied to the selective sensing of any class of analyte. The high sensitivity coupled with ease of selector attachment, means the technology could be used in the development of effective solid-state gas sensors.

### Example 4

MND Gas Sensing

The molecular nanolayer deposition (MND) process was applied with BocDBA to form aniline crosslinked multilayer films. The aniline crosslinked films were exposed to low concentrations of acetic acid vapour to assess the utility of the new architecture in solid state gas sensors.

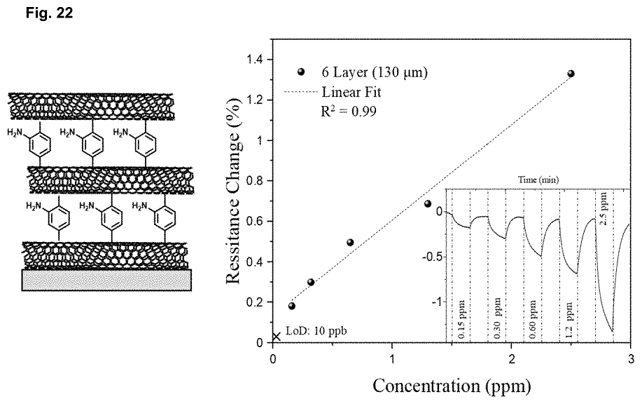

A 6 layer MND film displayed high sensitivity, eliciting a 1.3% resistance change when exposed to 2.5 ppm AcOH (FIG. 22). The device exhibited a significant resistance response of 0.18% at low AcOH concentration of 0.16 ppm, with minimal signal noise. The resistance response was highly linear over the measured concentration range. The sensing response for this device was used to plot the linear range, allowing for determination of the LoD. The high sensitivity and low noise resulted in a theoretical detection limit of 10 ppb, far surpassing the odour threshold. The response was also highly reversible with the initial resistance almost completely recovered following a six minute cycle of pure nitrogen.

Values disclosed herein as ends of ranges are not to be understood as being strictly limited to the exact numerical values recited. Instead, unless otherwise specified, each numerical range is intended to mean both the recited values, any integers within the specified range, and any ranges with the specified range. For example a range disclosed as “1 to 10” is intended to mean “1, 2, 3, 4, 5, 6, 7, 8, 9, and 10.” It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Values disclosed herein as ends of ranges are not to be understood as being strictly limited to the exact numerical values recited. Instead, unless otherwise specified, each numerical range is intended to mean both the recited values, any integers within the specified range, and any ranges with the specified range. For example a range disclosed as “1 to 10” is intended to mean “1, 2, 3, 4, 5, 6, 7, 8, 9, and 10.”

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system for utilizing a consumer product, the system comprising:
a central communication unit capable of receiving incoming signals and sending outgoing instructions, the central communication unit communicably connected with a memory configured to store an algorithm;
a sensor communicably connectable with the central communication unit and configured to send incoming signals to the central communication unit alerting the central communication unit of an identification and concentration of a target gas of interest, the sensor comprising a cross-linked carbon nanotube network comprising: a plurality of carbon nanotubes; and at least one linker that covalently links adjacent carbon nanotubes; and
a consumer product capable of boosting or mitigating the target gas of interest.

2. The system of claim 1, wherein the consumer product is communicably connectable with the central communication through a wireless communication link, wherein the algorithm controls the consumer product using incoming signals sent from the sensor to the central communication unit.

3. The system of claim 1, wherein the consumer product is selected from the group consisting of: an air freshener device, an air cleaning device, a tooth brush, a razor, a diaper, a feminine care product, a cleaning implement, and combinations thereof.

4. The system of claim 1, wherein the plurality of carbon nanotubes are single-walled carbon nanotubes.

5. The system of claim 1, wherein the plurality of carbon nanotubes are linked in a series of parallel layers, optionally wherein the cross-linked carbon nanotube has an in-plane conductivity that is greater than the through-thickness conductivity.

6. The system of claim 1, wherein the linker is a conjugated linker having a moiety of structure *-A-*, wherein A is a divalent conjugated system comprising one or more aryl or heteroaryl rings and * is the point of attachment to the carbon nanotubes.

7. The system of claim 6, wherein A is: 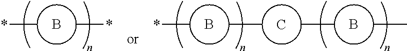
wherein
each Ring B is independently an optionally substituted aryl or heteroaryl;
Ring C is an optionally substituted porphyrin ring;
n is an integer from 1 to 5; and
* indicates the point of attachment to each X;
optionally wherein each Ring B may independently be 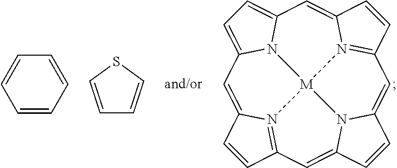
each of which may be optionally substituted, and wherein M is Zn, Cu, Ni or Co.

8. The system of claim 7, wherein the one or more aryl or heteroaryl rings are substituted with one or more of C₁-C₂₀alkyl, amino (including alkylamino and dialkylamino), carboxylic acid, amino-C₁-C₆alkyl, C₁-C₆alkyl-COOH, and —NHC(S)(NH₂), or a substituent comprising a Noble metal nanoparticle, a porphyrin, a calix[4]arenes or a crown ether.

9. The system of claim 1, wherein the linker is a conjugated linker having a moiety of structure: 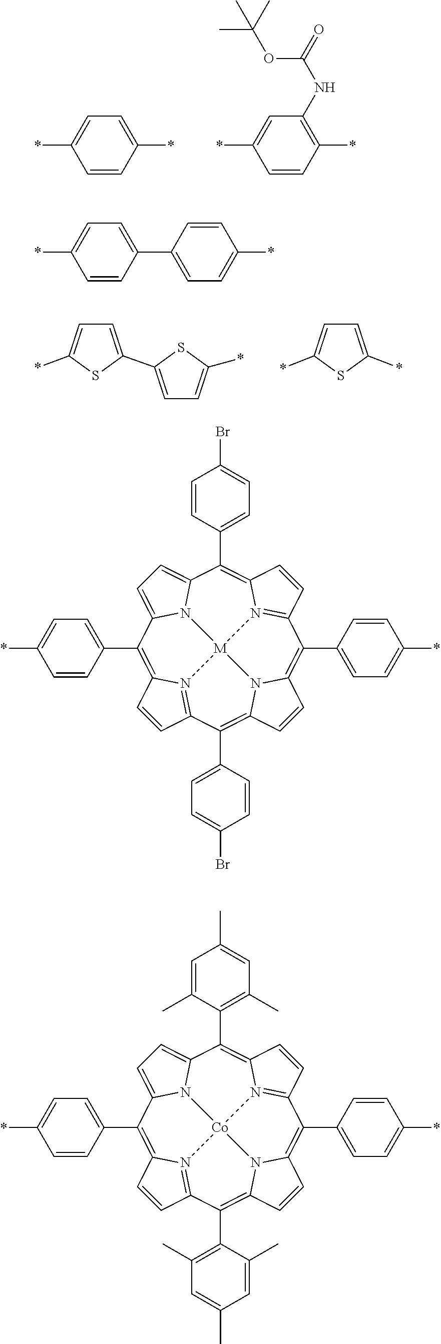 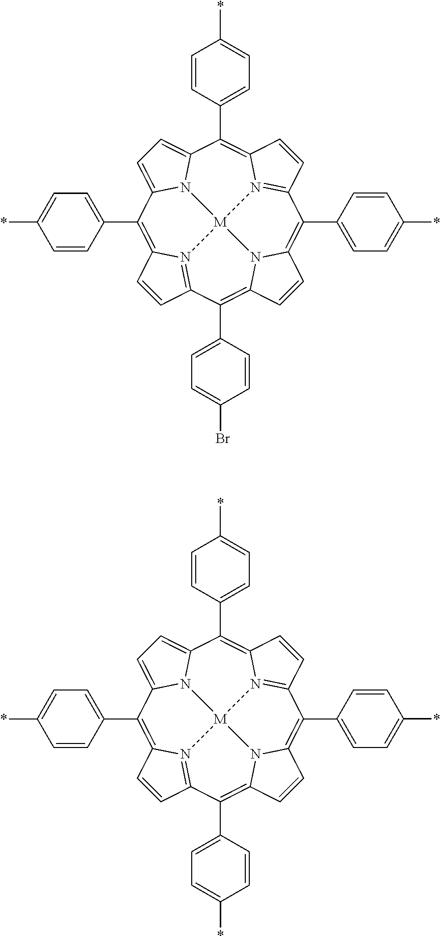
wherein M is Zn, Cu, Ni or Co.

10. The system of claim 1, wherein the linker is a rigid linker having a moiety of structure *—Y—*, wherein Y is a multivalent rigid system and * is the point of attachment to the carbon nanotubes.

11. The system of claim 10, wherein Y comprises a cycloalkyl group or a polyoctahedral silsequioxane.

12. The system of claim 1, wherein the plurality of carbon nanotubes form a film, wherein the film is provided on a substrate.

13. The system of claim 12, wherein the film has a thickness of about 1 to about 500 nm.

14. The system of claim 1, wherein the wireless communication link is selected from the group consisting of: Wi-Fi; Bluetooth; ZigBee, 6LoWPAN, Thread, Mesh Network, or combinations thereof.

15. The system of claim 1 further comprising an additional sensor that measures temperature, relative humidity, indoor air quality, outdoor air quality, noise, human presence, motion, air velocity in room, particle concentration in the air, allergens and/or other air borne entities.
